(19) United States
(12) Patent Application Publication
(10) Pub. No.: US 2026/0217625 A1
(43) Pub. Date: Jul. 30, 2026

(54) A METHOD FOR PREPARATION OF AN OLEFIN FROM AN ALCOHOL

(71) Applicant: BASE SE, Ludwigshafen am Rhein (DE)

(72) Inventors: Chiara BOSCAGLI, Hamburg (DE); Alexander CZAJA, Heidelberg (DE); Christian GRUENANGER, Ludwigshafen am Rhein (DE); Martin ERNST, Ludwigshafen am Rhein (DE); Alois KINDLER, Ludwigshafen am Rhein (DE); Michael Ludwig LEJKOWSI, Ludwigshafen am Rhein (DE); Piyush INGALE, Heidelberg (DE)

(21) Appl. No.: 19/142,992

(22) PCT Filed: Dec. 22, 2023
(86) PCT No.: PCT/EP2023/087637

§ 371 Date: Jun. 25, 2025

(30) Foreign Application Priority Data
Dec. 26, 2022 (EP) 22216680.3

(51) Int. Cl. C07C 1/20

(52) U.S. Cl. CPC C07C 1/20; C07C 2521/06; C07C 2523/62

(57) ABSTRACT

The presently claimed invention is directed to a method for preparing at least one olefin of formula (I),

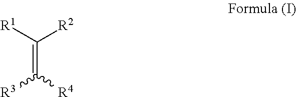

wherein, R¹, R², R³ and R⁴ are independently selected from hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, substituted C₂-C₂₀ alkynyl, C₆-C₂₀ aryl, substituted C₆-C₂₀ aryl, C₅-C₂₀ cycloalkyl, substituted C₅-C₂₀ cycloalkyl, C₇-C₂₀ arylalkyl, or substituted C₇-C₂₀ arylalkyl; or R¹ and R², together with the carbon to which they are attached, form a C₅-C₁₅ cycle, the method comprising:
a step of contacting at least one alcohol of formula (II),

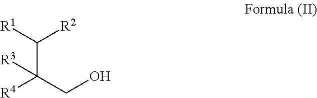

wherein, R¹, R², R³ and R⁴ are as defined above; with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4 bar, and at a temperature in the range of 200° C. to 450° C.

### FIELD OF THE INVENTION

Presently claimed invention is directed to a method for preparing an olefin by contacting an alcohol with a catalyst comprising at least one catalytically active metal selected from platinum (Pt), palladium (Pd), nickel (Ni), Chromium (Cr), Molybdenum (Mo), Ruthenium (Ru), or combinations thereof.

### BACKGROUND OF THE INVENTION

Hydroformylation reaction or “Oxo-process” involves addition of carbon monoxide and hydrogen (H₂) to an unsaturated carbon-carbon double bond of an unsaturated hydrocarbon to prepare an aldehyde compound. On the contrary, reverse-hydroformylation or retro-hydroformylation or dehydroformylation reaction involves conversion of an aldehyde into a corresponding olefin by eliminating syngas (carbon monoxide and dihydrogen).

For sustainable development of chemical industry, it is important to develop sustainable and efficient processes for converting renewable biomass derivatives into value-added chemicals like monomers, solvents, intermediate chemicals, and other fine chemicals. This includes catalytic conversion of a large feedstock of renewable oxygenated biomass derivatives into valuable bulk monomers such as olefins. There is, therefore, a need for continued development of new and sustainable catalytic processes for conversion of renewable oxygenated biomass derivatives such as alcohols and/or aldehydes into valuable bulk monomers such as olefins.

It is well known that catalytic processes for conversion of an alcohol into an olefin involves dehydration reactions (β-elimination reaction). However, alcohol and olefin have same number of carbon atoms.

Processes for conversion of aldehydes into olefins are known in the art.

Kusumoto S., et al (Angew. Chem. Int. Ed. 2015, 54, 8458-8461) discloses retro-hydroformylation reaction of an aldehyde into an alkene and synthesis gas (a mixture of carbon monoxide and dihydrogen) in the presence of a cyclopentadienyl iridium catalyst.

However, the known processes disclose preparing an olefin from an aldehyde by retro-hydroformylation. Therefore, these processes require extra steps for preparing an olefin from an alcohol, thereby making them cumbersome and expensive.

Therefore, there is a need for providing an efficient and a single-step catalytic process that can convert an alcohol into an olefin.

Therefore, it is an object of the presently claimed invention to provide a method for preparing an olefin from an alcohol. It is desired that the method is simple, preferably single step.

### SUMMARY OF THE INVENTION

Surprisingly, it has been found that the presently claimed invention provides a method for preparing an olefin by contacting an alcohol with a catalyst. The method is simple and efficient.

Thus, in an aspect, the presently claimed invention is directed to a method for preparing at least one olefin of formula (I),

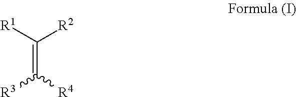

wherein, R¹, R², R³ and R⁴ are independently selected from hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, substituted C₂-C₂₀ alkynyl, C₆-C₂₀ aryl, substituted C₆-C₂₀ aryl, C₅-C₂₀ cycloalkyl, substituted C₅-C₂₀ cycloalkyl, C₇-C₂₀ arylalkyl, or substituted C₇-C₂₀ arylalkyl; or R¹ and R², together with the carbon to which they are attached, form a C₅-C₁₅ cycle,
the method comprising:
a step of contacting at least one alcohol of formula (II),

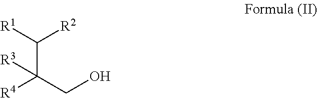

wherein, R¹, R², R³ and R⁴ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

### BRIEF DESCRIPTION OF THE DRAWINGS

FIG. 1, graphically illustrates conversion of isoamyl alcohol vs temperature using 0.25 ml of 1.0 wt % Pt/0.5 wt % Sn/ZrO₂ as catalyst, as represented in Table 2.

FIG. 2, graphically illustrates conversion of isoamyl alcohol vs temperature using 1.0 ml of 1.0 wt % Pt/0.5 wt % Sn/ZrO₂ as catalyst, as represented in Table 3.

FIG. 3, graphically illustrates conversion of isoamyl alcohol vs temperature using 1.0 ml of 1.0 wt % Pt/0.5 wt % Sn/ZrO₂ as catalyst at a constant GHSV of 2500 h⁻¹, as represented in Table 4.

FIG. 4, graphically illustrates conversion of isoamyl alcohol vs temperature using 0.25 ml of 1.0 wt % Pt/0.5 wt % Sn/ZrO₂ as catalyst at a constant GHSV of 9800 h⁻¹, as represented in Table 5.

FIG. 5, graphically illustrates conversion of isoamyl alcohol vs % of isoamyl alcohol using 1.0 ml of 1.0 wt % Pt/0.5 wt % Sn/ZrO₂ as catalyst, at various GHSV's, at Temp=300° C., and Pressure=1 bar, as represented in Table 6.

FIG. 6, graphically illustrates conversion of isoamyl alcohol vs temperature using 0.25 ml of Nickel (60 wt % metal content, Total Pore Volume 0.45 cm³/g) as catalyst, as represented in Table 7.

FIG. 7, graphically illustrates conversion of isoamyl alcohol vs temperature using 1.0 ml of Nickel (60 wt % metal content, Total Pore Volume 0.45 cm³/g) as catalyst, as represented in Table 8.

FIG. 8, graphically illustrates conversion of isoamyl alcohol vs temperature using 1.0 ml of Nickel (56 wt % metal content, Total Pore Volume 0.3 cm³/g) as catalyst, as represented in Table 9.

FIG. 9, graphically illustrates conversion of isoamyl alcohol vs temperature using 1.0 ml Nickel-Copper as catalyst, as represented in Table 10.

FIG. 10, graphically illustrates conversion of isoamyl alcohol vs temperature using 0.25 ml of Nickel-Copper as catalyst, as represented in Table 11.

FIG. 11, graphically illustrates conversion of isoamyl alcohol vs temperature using 1.0 ml of Nickel-Copper as catalyst, as represented in Table 12.

FIG. 12, graphically illustrates conversion of isoamyl alcohol vs temperature using 1.0 ml Nickel-Copper as catalyst, as represented in Table 13.

FIG. 13, graphically illustrates conversion of isoamyl alcohol vs temperature using 0.5 ml of 1.0 wt % Pt/0.5 wt % Sn/ZrO₂+Cu-Zn as catalyst, as represented in Table 14.

FIG. 14, graphically illustrates conversion of isoamyl alcohol vs temperature using 0.5 ml of 1.0 wt % Pt/0.5 wt % Sn/ZrO₂+Cu-Zn as catalyst, as represented in Table 15.

FIG. 15, graphically illustrates conversion of isoamyl alcohol vs temperature using 1.0 ml of Ru as catalyst, as represented in Table 16.

FIG. 16, graphically illustrates conversion of isoamyl alcohol vs temperature using 1.0 ml of 0.3% Pd as catalyst, as represented in Table 17.

FIG. 17, graphically illustrates conversion of isoamyl alcohol vs temperature using 1.0 ml of 0.5% Pd as catalyst, as represented in Table 18.

FIG. 18, graphically illustrates conversion of isoamyl alcohol vs temperature using 1.0 ml of (Pd) as catalyst, as represented in Table 19.

### DETAILED DESCRIPTION

Before the method of the presently claimed invention is described, it is to be understood that this invention is not limited to particular method described, since such method may, of course, vary. It is also to be understood that the terminology used herein is not intended to be limiting, since the scope of the presently claimed invention will be limited only by the appended claims.

If hereinafter a group is defined to comprise at least a certain number of embodiments, this is meant to also encompass a group which preferably consists of these embodiments only. Furthermore, the terms ‘first’, ‘second’, ‘third’ or ‘a’, ‘b’, ‘c’, etc. and the like in the description and in the claims, are used for distinguishing between similar elements and not necessarily for describing a sequential or chronological order. It is to be understood that the terms so used are interchangeable under appropriate circumstances and that the embodiments of the presently claimed invention described herein are capable of operation in other sequences than described or illustrated herein. In case the terms ‘first’, ‘second’, ‘third’ or ‘(A)’, ‘(B)’ and ‘(C)’ or ‘(a)’, ‘(b)’, ‘(c)’, ‘(d)’, ‘i’, ‘ii’ etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

Furthermore, the ranges defined throughout the specification include the end values as well i.e., a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to ‘one embodiment’ or ‘an embodiment’ means that a particular feature, structure or characteristic described in connection with the embodiment is included in at least one embodiment of the presently claimed invention. Thus, appearances of the phrases ‘in one embodiment’ or ‘in an embodiment’ in various places throughout this specification are not necessarily all referring to the same embodiment but may refer to so.

Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any of the claimed embodiments can be used in any combination.

The presently claimed invention provides a one-step catalytic process for preparing an olefin from an alcohol.

In one aspect, the presently claimed invention is directed to a method for preparing at least one olefin of formula (I),

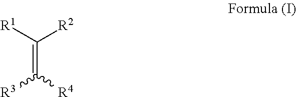

wherein, R¹, R², R³ and R⁴ are independently selected from hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, substituted C₂-C₂₀ alkynyl, C₆-C₂₀ aryl, substituted C₆-C₂₀ aryl, C₅-C₂₀ cycloalkyl, substituted C₅-C₂₀ cycloalkyl, C₇-C₂₀ arylalkyl, or substituted C₇-C₂₀ arylalkyl; or R¹ and R², together with the carbon to which they are attached, form a C₅-C₁₅ cycle, the method comprising a step of contacting at least one alcohol of formula (II),

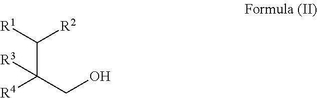

wherein, R¹, R², R³ and R⁴ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

Preferably, the method for preparing at least one olefin of formula (I),

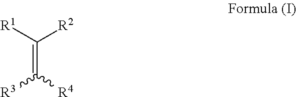

wherein, R¹, R², R³ and R⁴ are independently selected from hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, substituted C₂-C₂₀ alkynyl, C₆-C₂₀ aryl, substituted C₆-C₂₀ aryl, C₅-C₂₀ cycloalkyl, substituted C₅-C₂₀ cycloalkyl, C₇-C₂₀ arylalkyl, or substituted C₇-C₂₀ arylalkyl; or R¹ and R², together with the carbon to which they are attached, form a C₅-C₁₅ cycle,
wherein each R¹, R², R³ and R⁴, when substituted, independently bear one, two, three or four substituents which are selected from —OH, —OR⁵, —C(═O)R⁵, —COOR⁵, —O—C(═O)R⁵, O—C(═O)—OR⁵, —O—C(═O)—NR⁵R⁶, —NR⁵—C(═O)—R⁶, —NR⁵—C(═O)—OR⁶, —NR⁵—C(═O)—NR⁶R⁷, nitro or cyano, wherein R⁵, R⁶ and R⁷ are each independently hydrogen, alkyl, cycloalkyl, heterocycloalkyl, aryl or heteroaryl,
the method comprising a step of contacting at least one alcohol of formula (II),

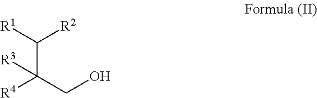

wherein, R¹, R², R³, R⁴, R⁵, R⁶ and R⁷ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, at a temperature in the range of 200° C. to 450° C.

Preferably, the method for preparing at least one olefin of formula (I),

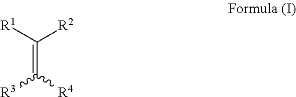

wherein, R¹, R², R³ and R⁴ are independently selected from hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, substituted C₂-C₂₀ alkynyl, C₆-C₂₀ aryl, substituted C₆-C₂₀ aryl, C₅-C₂₀ cycloalkyl, substituted C₅-C₂₀ cycloalkyl, C₇-C₂₀ arylalkyl, or substituted C₇-C₂₀ arylalkyl; or R¹ and R², together with the carbon to which they are attached, form a C₅-C₁₅ cycle,
wherein each R¹, R², R³ and R⁴, when substituted, independently bear one, two, three or four substituents which are selected from —OH, —OR⁵, —C(═O)R⁵, —COOR⁵, —O—C(═O)R⁵, O—C(═O)—OR⁵ or cyano, wherein R⁵ is hydrogen, alkyl, cycloalkyl, heterocycloalkyl, aryl or heteroaryl,
the method comprising a step of contacting at least one alcohol of formula (II),

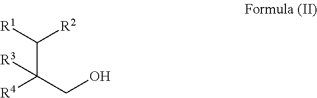

wherein, R¹, R², R³, R⁴ and R⁵ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

Preferably, the method for preparing at least one olefin of formula (I),

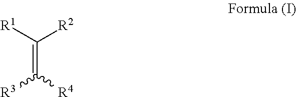

wherein, R¹, R², R³ and R⁴ are independently selected from hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, substituted C₂-C₂₀ alkynyl, C₆-C₂₀ aryl, substituted C₆-C₂₀ aryl, C₅-C₂₀ cycloalkyl, substituted C₅-C₂₀ cycloalkyl, C₇-C₂₀ arylalkyl, or substituted C₇-C₂₀ arylalkyl; or R¹ and R², together with the carbon to which they are attached, form a C₅-C₁₅ cycle, wherein each R¹, R², R³ and R⁴, when substituted, independently bear one, two, three or four substituents which are selected from —OH, —OR⁵, wherein R⁵ is hydrogen, alkyl, cycloalkyl, heterocycloalkyl, aryl or heteroaryl, the method comprising a step of contacting at least one alcohol of formula (II),

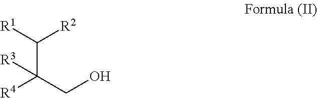

wherein, R¹, R², R³, R⁴ and R⁵ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

Preferably, the method for preparing at least one olefin of formula (I),

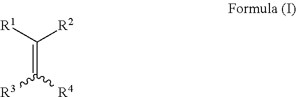

wherein, R¹, R², R³ and R⁴ are independently selected from hydrogen, C₁-C₂₀ alkyl, C₂-C₂₀ alkenyl, C₂-C₂₀ alkynyl, C₆-C₂₀ aryl, C₅-C₂₀ cycloalkyl, or C₇-C₂₀ arylalkyl; or R¹ and R², together with the carbon to which they are attached, form a C₅-C₁₅ cycle, the method comprising a step of contacting at least one alcohol of formula (II),

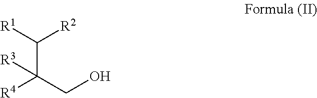

wherein, R¹, R², R³ and R⁴ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

The term “alkyl” as used herein, refers to a saturated, linear or branched hydrocarbon radical containing between 1 to 20 carbon atoms that can be obtained by removal of a single hydrogen atom from an aliphatic moiety. Unless otherwise specified, alkyl group contains 1-20 carbon atoms. In certain embodiments, alkyl group contains 1-16 carbon atoms. In certain embodiments, alkyl group contains 1-12 carbon atoms. In some embodiments, alkyl group contains 1-8 carbon atoms, in some embodiments, alkyl group contains 1-4 carbon atoms, in some embodiments alkyl group contains 1-3 carbon atoms, and in some embodiments alkyl group contains 1-2 carbon atoms. Examples of alkyl radical include, but are not limited to methyl, ethyl, n-propyl, isopropyl, n-butyl, iso-butyl, sec-butyl, tert-butyl, sec-pentyl, iso-pentyl, n-pentyl, neopentyl, n-hexyl, sec-hexyl, n-heptyl, n-octyl, n-decyl, n-undecyl, dodecyl, and the like.

The term “alkenyl” as used herein, refers to a monovalent, unsaturated hydrocarbon radical having at least one carbon-carbon double bond that can be obtained from a linear or branched unsaturated moiety by removal of a single hydrogen atom. Unless otherwise specified, alkenyl group contains 2-20 carbon atoms. In certain embodiments, alkenyl group contains 2-16 carbon atoms. In certain embodiments, alkenyl group contains 2-12 carbon atoms. In some embodiments, alkenyl group contains 2-8 carbon atoms, in some embodiments, alkenyl group contains 2-4 carbon atoms, in some embodiments alkenyl group contains 2-3 carbon atoms, and in some embodiments alkenyl group contains 2 carbon atoms. Possible alkenyl group includes, for example, ethenyl, propenyl, butenyl, 1-methyl-2-buten-1-yl, and the like.

The term “alkynyl” as used herein, refers to a monovalent, unsaturated hydrocarbon radical having at least one carbon-carbon triple bond that can be obtained from a linear or branched unsaturated moiety by removal of a single hydrogen atom. Unless otherwise specified, alkynyl groups contain 2-20 carbon atoms. In certain embodiments, alkynyl group contains 2-16 carbon atoms. In certain embodiments, alkynyl group contains 2-12 carbon atoms. In some embodiments, alkynyl group contains 2-8 carbon atoms, in some embodiments, alkynyl group contains 2-4 carbon atoms, in some embodiments alkynyl group contains 2-3 carbon atoms, and in some embodiments alkynyl group contains 2 carbon atoms. Representative alkynyl group includes, but is not limited to, ethynyl, 2-propynyl (propargyl), 1-propynyl, and the like.

As described herein, any compound of the present invention may, when particularly specified, contain “substituted” moieties. In general, the term “substituted” means at least one substitutable hydrogen of the compound is replaced with a suitable substituent. Unless otherwise indicated, “substituted” compound may have a suitable substituent at each substitutable position of the compound, and when more than one hydrogen in any given compound may be substituted with more than one substituents selected from a specified group, the substituent may be either the same or different at every position. Combinations of the substituents envisioned by this invention are preferably those that result in the formation of stable or chemically feasible compounds. Suitable monovalent substituents can independently be —OH, —OR^{a}, —C(═O)R^{a}, —COOR^{a}, —OC(═O)R^{a}, O—C(═O)—OR^{a}, —O—C(═O)—NR^{a}R^{b}, —NR^{a}—C(═O)—R^{b}, —NR^{a}—C(═O)—OR^{b}, —NR^{a}—C(═O)—NR^{b}R^{c}, nitro and cyano, wherein R^{a}, R^{b} and R^{c} are each independently hydrogen, alkyl, cycloalkyl, heterocycloalkyl, aryl or heteroaryl.

The term “cycloalkyl” as used herein, refers to a monovalent, cyclic hydrocarbon ring system having a total of 5 to 20 carbon atoms, which is completely saturated or contains one or more units of unsaturation, but which is not aromatic.

The term “aryl” as used herein, refers to a monovalent, cyclic ring system having a total of 5 to 20 carbon atoms, wherein at least one ring in the system that is aromatic and wherein each ring in the system contains five to seven carbon atoms. In certain embodiments of the present invention, “aryl” refers to an aromatic ring system which includes, but is not limited to phenyl, biphenyl, naphthyl, anthracenyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” as used herein, refers to a monovalent, cyclic ring system having a total of 5 to 20 carbon atoms, wherein at least one ring in the system is aromatic and wherein each ring in the system contains five to seven carbon atoms and having 6, 10, or 14 electrons shared in a cyclic array; and having, in addition to carbon atoms, from one to five heteroatoms. Examples of the heteroaryl includes, without limitation, furanyl, pyrrolyl, imidazolyl, pyrazolyl, pyridyl, and the like. The terms “heteroaryl” as used herein, also includes compounds in which a heteroaromatic ring is fused to one or more aryl, cycloaliphatic, or heterocyclyl rings, where the radical or point of attachment is on the heteroaromatic ring.

The terms “heterocycle” as used herein, refers to a monovalent, cyclic hydrocarbon ring system having a total of 5 to 20 carbon atoms, wherein each of the rings is having 5-, 6- or 7-carbon atoms and containing one, two, three, or four heteroatoms independently selected from the group consisting of nitrogen, oxygen, and sulfur, independently each ring is completely saturated or contains one or more units of unsaturation, but which is not aromatic. The term “heterocyclic” also includes a bridged or fused bicyclic, tricyclic, tetracyclic and multicyclic structures in which one or more carbons and/or heteroatoms bridges two non-adjacent members of a monocyclic ring. Examples of heterocycles include pyrrolyl, pyrazolyl, imidazolyl, imidazolinyl, imidazolidinyl, pyridyl, piperidinyl, and the like.

The term “unsaturated,” as used herein, means that a moiety has one or more units of unsaturation i.e., carbon-carbon double bond or carbon-carbon triple bond.

In a preferred embodiment, the method for preparing at least one olefin of formula (Ia),

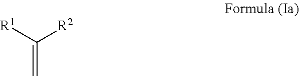

wherein, R¹ and R² are independently selected from hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, substituted C₂-C₂₀ alkynyl, C₆-C₂₀ aryl, substituted C₆-C₂₀ aryl, C₅-C₂₀ cycloalkyl, substituted C₅-C₂₀ cycloalkyl, C₇-C₂₀ arylalkyl, or substituted C₇-C₂₀ arylalkyl; or R¹ and R², together with the carbon to which they are attached, form a C₅-C₁₅ cycle,
the method comprising a step of contacting at least one alcohol of formula (IIa),

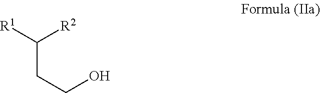

wherein, R¹ and R² are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

Preferably, the method for preparing at least one olefin of formula (Ia),

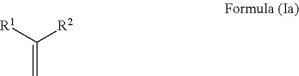

wherein, R¹ and R² are independently selected from hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, substituted C₂-C₂₀ alkynyl, C₆-C₂₀ aryl, substituted C₆-C₂₀ aryl, C₅-C₂₀ cycloalkyl, substituted C₅-C₂₀ cycloalkyl, C₇-C₂₀ arylalkyl, or substituted C₇-C₂₀ arylalkyl; or R¹ and R², together with the carbon to which they are attached, form a C₅-C₁₅ cycle,
wherein each R¹ and R², when substituted, independently bear one, two, three or four substituents which are selected from —OH, —OR⁵, —C(═O)R⁵, —COOR⁵, —O—C(═O)R⁵, O—C(═O)—OR⁵, —O—C(═O)—NR⁵R⁶, —NR⁵—C(═O)—R⁶, —NR⁵—C(═O)—OR⁶, —NR⁵—C(═O)—NR⁶R⁷, nitro or cyano, wherein R⁵, R⁶ and R⁷ are each independently hydrogen, alkyl, cycloalkyl, heterocycloalkyl, aryl or heteroaryl,
the method comprising a step of contacting at least one alcohol of formula (IIa),

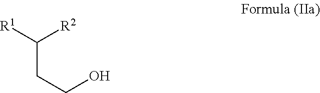

wherein, R¹, R², R⁵, R⁶ and R⁷ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

Preferably, the method for preparing at least one olefin of formula (Ia),

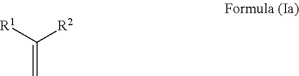

wherein, R¹ and R² are independently selected from hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, substituted C₂-C₂₀ alkynyl, C₆-C₂₀ aryl, substituted C₆-C₂₀ aryl, C₅-C₂₀ cycloalkyl, substituted C₅-C₂₀ cycloalkyl, C₇-C₂₀ arylalkyl, or substituted C₇-C₂₀ arylalkyl; or R¹ and R², together with the carbon to which they are attached, form a C₅-C₁₅ cycle,
wherein each R¹ and R², when substituted, independently bear one, two, three or four substituents which are selected from —OH, —OR⁵, —C(═O)R⁵, —COOR⁵, —O—C(═O)R⁵, O—C(═O)—OR⁵ and cyano, wherein R⁵, is hydrogen, alkyl, cycloalkyl, heterocycloalkyl, aryl or heteroaryl,
the method comprising a step of contacting at least one alcohol of formula (IIa),

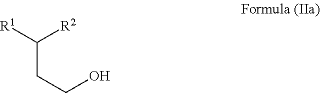

wherein, R¹, R² and R⁵ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

Preferably, the method for preparing at least one olefin of formula (Ia),

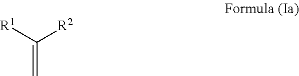

wherein, R¹ and R² are independently selected from hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, substituted C₂-C₂₀ alkynyl, C₆-C₂₀ aryl, substituted C₆-C₂₀ aryl, C₅-C₂₀ cycloalkyl, substituted C₅-C₂₀ cycloalkyl, C₇-C₂₀ arylalkyl, or substituted C₇-C₂₀ arylalkyl; or R¹ and R², together with the carbon to which they are attached, form a C₅-C₁₅ cycle,
wherein each R¹ and R², when substituted, independently bear one, two, three or four substituents which are selected from —OH, or —OR⁵, wherein R⁵, is hydrogen, alkyl, cycloalkyl, heterocycloalkyl, aryl or heteroaryl,
the method comprising a step of contacting at least one alcohol of formula (IIa),

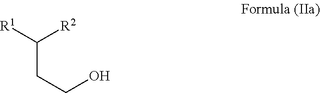

wherein, R¹, R² and R⁵ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

Preferably, the method for preparing at least one olefin of formula (Ia),

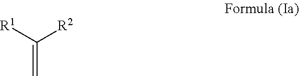

wherein, R¹ and R² are independently selected from hydrogen, C₁-C₂₀ alkyl, C₂-C₂₀ alkenyl, C₂-C₂₀ alkynyl, C₆-C₂₀ aryl, C₅-C₂₀ cycloalkyl, C₇-C₂₀ arylalkyl; or R¹ and R², together with the carbon to which they are attached, form a C₅-C₁₅ cycle,
the method comprising a step of contacting at least one alcohol of formula (IIa),

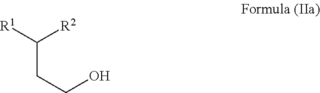

wherein, R¹ and R² are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

In another preferred embodiment, the method for preparing at least one olefin of formula (Ia),

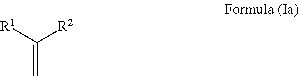

wherein, R¹ and R² are independently selected from hydrogen, C₁-C₂₀ alkyl, substituted C₁-C₂₀ alkyl,
the method comprising a step of contacting at least one alcohol of formula (IIa),

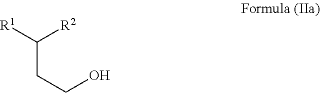

wherein, R¹ and R² are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

Preferably, the method for preparing at least one olefin of formula (Ia),

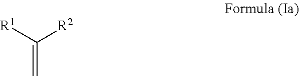

wherein, R¹ and R² are independently selected from hydrogen, C₁-C₂₀ alkyl, substituted C₁-C₂₀ alkyl,
wherein each R¹ and R², when substituted, independently bear one, two, three or four substituents which are selected from —OH, —OR⁵, —C(═O)R⁵, —COOR⁵, —O—C(═O)R⁵, O—C(═O)—OR⁵, —O—C(═O)—NR⁵R⁶, —NR₅—C(═O)—R⁶, —NR⁵—C(═O)—OR⁶, —NR⁵—C(═O)—NR⁶R⁷, nitro or cyano, wherein R⁵, R⁶ and R⁷ are each independently hydrogen, alkyl, cycloalkyl, heterocycloalkyl, aryl or heteroaryl,
the method comprising a step of contacting at least one alcohol of formula (IIa),

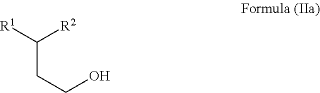

wherein, R¹, R², R⁵, R⁶ and R⁷ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

Preferably, the method for preparing at least one olefin of formula (Ia),

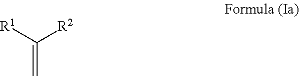

wherein, R¹ and R² are independently selected from hydrogen, C₁-C₂₀ alkyl, substituted C₁-C₂₀ alkyl,
wherein each R¹ and R², when substituted, independently bear one, two, three or four substituents which are selected from —OH, —OR⁵, —C(═O)R⁵, —COOR⁵, —O—C(═O)R⁵, O—C(═O)—OR⁵, or cyano, wherein R⁵ is hydrogen, alkyl, cycloalkyl, heterocycloalkyl, aryl or heteroaryl,
the method comprising a step of contacting at least one alcohol of formula (IIa),

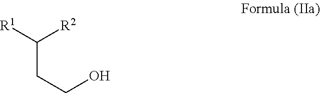

wherein, R¹, R² and R⁵ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

Preferably, the method for preparing at least one olefin of formula (Ia),

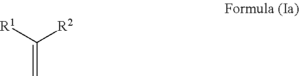

wherein, R¹ and R² are independently selected from hydrogen, C₁-C₂₀ alkyl, substituted C₁-C₂₀ alkyl,
wherein each R¹ and R², when substituted, independently bear one, two, three or four substituents which are selected from —OH, or —OR⁵, wherein R⁵ is hydrogen, alkyl, cycloalkyl, heterocycloalkyl, aryl or heteroaryl,
the method comprising a step of contacting at least one alcohol of formula (IIa),

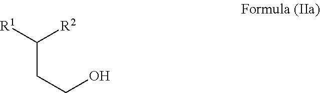

wherein, R¹, R² and R⁵ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

In another preferred embodiment, the method for preparing at least one olefin of formula (Ia),

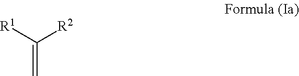

wherein, R¹ is C₁-C₂₀ alkyl, and R² is hydrogen,
the method comprising a step of contacting at least one alcohol of formula (IIa),

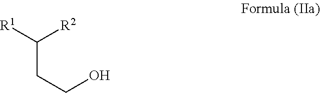

wherein, R¹ and R² are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

In a preferred embodiment, the method for preparing at least one olefin selected from ethene, propene, isobutene, butene, pentene, hexene, heptene, octene, nonene, or decene, comprises a step of contacting at least one alcohol selected from propanol, butanol, isoamyl alcohol, pentanol, hexanol, heptanol, octanol, nonanol, decanol, or undecanol, with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

In a preferred embodiment, the method for preparing at least one olefin selected from ethene, propene, or isobutene, comprises a step of contacting at least one alcohol selected from propanol, butanol, or isoamyl alcohol, with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

In a preferred embodiment, the method for preparing isobutene, comprises a step of contacting isoamyl alcohol, with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.

In a preferred embodiment, the at least one catalytically active metal is present in an amount in the range from 0.005 wt % to 10.0 wt %, with respect to total weight of the catalyst.

Preferably, the at least one catalytically active metal is present in an amount in the range from 0.005 wt % to 5.0 wt %, with respect to total weight of the catalyst.

More preferably, the at least one catalytically active metal is present in an amount in the range from 0.2 wt % to 2.0 wt %, with respect to total weight of the catalyst.

More preferably, the at least one catalytically active metal is present in an amount in the range from 0.5 wt % to 1.5 wt %, with respect to total weight of the catalyst.

More preferably, the at least one catalytically active metal is present in an amount of 1.0 wt %, with respect to total weight of the catalyst.

In a preferred embodiment, the at least one catalyst comprises
a. at least one catalytically active metal selected from Platinum (Pt), Palladium (Pd), Nickel (Ni), Chromium (Cr), Molybdenum (Mo), Ruthenium (Ru), or combinations thereof; and
b. at least one promoter selected from Tin (Sn), Silver (Ag), Iridium (Ir), Rhenium (Re), or combinations thereof, wherein the at least one promoter is present in an amount in the range from 0.005 wt % to 5.0 wt %, with respect to total weight of the catalyst.

Preferably, the at least one catalyst comprises
a. at least one catalytically active metal selected from Platinum (Pt), Palladium (Pd), Nickel (Ni), or combinations thereof; and
b. at least one promoter selected from Tin (Sn), Silver (Ag), Iridium (Ir), Rhenium (Re), or combinations thereof, wherein the at least one promoter is present in an amount in the range from 0.005 wt % to 5.0 wt %, with respect to total weight of the catalyst.

Preferably, the at least one catalyst comprises
a. at least one catalytically active metal selected from Platinum (Pt), Palladium (Pd), Nickel (Ni), or combinations thereof; and
b. at least one promoter selected from Tin (Sn), Rhenium (Re), or combinations thereof, wherein the at least one promoter is present in an amount in the range from 0.005 wt % to 5.0 wt %, with respect to total weight of the catalyst.

In a preferred embodiment, the at least one promoter is present in an amount in the range from 0.1 wt % to 3.0 wt %, with respect to total weight of the catalyst.

Preferably, the at least one promoter is present in an amount in the range from 0.2 wt % to 2.0 wt %, with respect to total weight of the catalyst.

Preferably, the at least one promoter is present in an amount in the range from 0.3 wt % to 1.5 wt %, with respect to total weight of the catalyst.

Preferably, the at least one promoter is present in an amount of 0.5 wt %, with respect to total weight of the catalyst.

In a preferred embodiment, the at least one catalyst comprises
a. at least one catalytically active metal selected from Platinum (Pt), Palladium (Pd), Nickel (Ni), Chromium (Cr), Molybdenum (Mo), Ruthenium (Ru), or combinations thereof; and
b. at least one carrier on which the catalytically active metal is supported, wherein the carrier is selected from a non-acidic material selected from calcium oxide, titanium oxide, beryllia, magnesia, thoria, zirconia, alumina, silicon carbide, quartz or combinations thereof, and
   wherein the at least one carrier is present in an amount in the range from 90.0 wt % to 99.995 wt %, with respect to total weight of the catalyst.

Preferably, the at least one catalyst comprises
a. at least one catalytically active metal selected from Platinum (Pt), Palladium (Pd), Nickel (Ni), or combinations thereof; and
b. at least one carrier on which the catalytically active metal is supported, wherein the carrier is selected from a non-acidic material selected from calcium oxide, titanium oxide, beryllia, magnesia, thoria, zirconia, alumina, silicon carbide, quartz or combinations thereof, and
   wherein the at least one carrier is present in an amount in the range from 90.0 wt % to 99.995 wt %, with respect to total weight of the catalyst.

Preferably, the at least one catalyst comprises
a. at least one catalytically active metal selected from Platinum (Pt), Palladium (Pd), Nickel (Ni), or combinations thereof; and
b. at least one carrier on which the catalytically active metal is supported, wherein the carrier is selected from a non-acidic material selected from titanium oxide, zirconia alumina, or combinations thereof, and
   wherein the at least one carrier is present in an amount in the range from 90.0 wt % to 99.995 wt %, with respect to total weight of the catalyst.

In a preferred embodiment, the at least one catalyst comprises
a. at least one catalytically active metal selected from Platinum (Pt), Palladium (Pd), Nickel (Ni), Chromium (Cr), Molybdenum (Mo), Ruthenium (Ru), or combinations thereof;
b. at least one promoter selected from Tin (Sn), Silver (Ag), Iridium (Ir), Rhenium (Re), or combinations thereof; and
c. at least one carrier on which the catalytically active metal is supported, wherein the carrier is selected from a non-acidic material selected from calcium oxide, titanium oxide, beryllia, magnesia, thoria, zirconia, alumina, silicon carbide, quartz or combinations thereof.

Preferably, the at least one catalyst comprises
a. at least one catalytically active metal selected from Platinum (Pt), Palladium (Pd), Nickel (Ni), or combinations thereof;
b. at least one promoter selected from Tin (Sn), Silver (Ag), Iridium (Ir), Rhenium (Re), or combinations thereof; and
c. at least one carrier on which the catalytically active metal is supported, wherein the carrier is selected from a non-acidic material selected from calcium oxide, titanium oxide, beryllia, magnesia, thoria, zirconia, alumina, silicon carbide, quartz or combinations thereof.

Preferably, the at least one catalyst comprises
a. at least one catalytically active metal selected from Platinum (Pt), Palladium (Pd), Nickel (Ni), or combinations thereof;
b. at least one promoter selected from Tin (Sn), Rhenium (Re), or combinations thereof; and
c. at least one carrier on which the catalytically active metal is supported, wherein the carrier is selected from a non-acidic material selected from titanium oxide, zirconia, alumina, or combinations thereof.

In a preferred embodiment, the at least one catalyst comprises at least one catalytically active metal Platinum (Pt), at least one promoter Tin (Sn), and at least one carrier Zirconia (ZrO₂).

In the present invention the crystalline phase of the carrier is stable under the conditions of the reaction involving conversion of an alcohol into an aldehyde followed by reverse-hydroformylation or retro-hydroformylation or dehydroformylation reaction involving conversion of an aldehyde into a corresponding olefin by eliminating syngas (carbon monoxide and dihydrogen).

The preparation of the at least one catalyst comprising at least one catalytically active metal Platinum (Pt), at least one promoter Tin (Sn), and at least one carrier Zirconia (ZrO₂) of the present invention, as disclosed in U.S. Pat. No. 6,989,346 B2 is specifically incorporated by reference herein.

In another preferred embodiment, the at least one catalyst further comprises other possible elements that are known to influence the acidity of the catalyst surface and/or to stabilize the catalytically active metals against sintering. For example, elements of main groups I and II, i.e. Li, Na, K, R^{b}, Cs on the one hand and Mg, Ca, Sr and Ba on the other hand, elements of main group III i.e. gallium, indium and thallium, and elements of transition group III i.e. Y and La and also rare earth elements. When tetragonal ZrO₂ is employed as a carrier, it can be stabilized by doping with Lanthanum (La) or Yttrium (Y). Zinc has also been found to be effective.

In another preferred embodiment the at least one catalyst has a BET surface area in the range from 1 to 500 square meters per gram (m²/g), as measured by the Brunauer-Emmett-Teller method.

Preferably, the at least one catalyst has a BET surface area in the range from 5 to 300 square meters per gram (m²/g), as measured by the Brunauer-Emmett-Teller method.

More preferably, the at least one catalyst has a BET surface area in the range from 10 to 200 square meters per gram (m²/g), as measured by the Brunauer-Emmett-Teller method.

More preferably, the at least one catalyst has a BET a surface area in the range from 20 to 100 square meters per gram (m²/g), as measured by the Brunauer-Emmett-Teller method.

In the presently claimed invention, the amount of the at least one catalytically active metal that can be effectively disposed upon a suitable carrier varies depending usually upon the surface area of the carrier.

The amount of the at least one promoter present in the catalyst varies depending upon the at least one catalytically active metal and the hydrocarbon feed employed.

Promoter has been found to help inhibit isomerization and cracking of hydrocarbon feed during the high temperature catalytic processes involving precious metals such as Platinum and Palladium. Promoter has also been found to stabilize the catalyst and extend its life. The catalyst can be regenerated by conventional oxidation of the catalyst.

In a preferred embodiment, the at least one carrier support is non-acidic refractory material selected from calcium oxide, titanium oxide, beryllia, magnesia, thoria, zirconia, alumina, silica, silicon carbide, quartz or combinations thereof Preferably, the at least one carrier support is non-acidic refractory material selected from calcium oxide, titanium oxide, magnesia, zirconia, alumina, silica, or combinations thereof.

More preferably, the at least one carrier support is non-acidic refractory material selected from magnesia, zirconia, titanium oxide, alumina, silica, or combinations thereof.

More preferably, the at least one carrier support is non-acidic refractory material zirconia.

In a preferred embodiment, the at least one carrier is present in an amount in the range from 95.0 wt % to 99.5 wt %, with respect to total weight of the catalyst.

Preferably, the at least one carrier is present in an amount in the range from 97.0 wt % to 99.0 wt %, with respect to total weight of the catalyst.

Preferably, the at least one carrier is present in an amount of 98.5 wt %, with respect to total weight of the catalyst.

In a preferred embodiment, the at least one catalyst has a pore volume in the range from 0.1 to 1.0 ml/g, as determined by Mercury (Hg) Porosimetry.

Preferably, the at least one catalyst has a pore volume in the range from 0.15 to 0.6 ml/g, as determined by Mercury (Hg) Porosimetry.

More preferably, the at least one catalyst has a pore volume in the range from 0.2 to 0.4 ml/g, as determined by Mercury (Hg) Porosimetry.

In a preferred embodiment, the at least one catalyst has a mean pore diameter, as determined by Mercury (Hg) Porosimetry, in the range from 0.008 to 0.06 microns (μ).

Preferably, the at least one catalyst has a mean pore diameter, as determined by Mercury (Hg) Porosimetry, in the range from 0.01 to 0.04 microns (μ).

In a preferred embodiment, the step of contacting is carried out at a temperature in the range from 250° C. to 450° C.

In a preferred embodiment, the at least one catalyst comprises at least one catalytically active metal selected from Platinum (Pt), Palladium (Pd), Ruthenium (Ru), or combinations thereof, and the step of contacting is carried out at a temperature in the range from 250° C. to 450° C., preferably 300 to 450° C.

In a preferred embodiment, the at least one catalyst comprises at least one catalytically active metal selected from Platinum (Pt), Palladium (Pd), or combinations thereof, at least one promoter, and at least one carrier, and the step of contacting is carried out at a temperature in the range from 250° C. to 450° C., preferably 300 to 450° C.

Preferably, the step of contacting is carried out at a temperature in the range from 300° C. to 400° C.

Preferably, the step of contacting is carried out at a temperature in the range from 300° C. to 350° C.

In a preferred embodiment, the at least one catalytically active metal comprises Nickel (Ni). Preferably, the catalyst has a Nickel content in the range from 10 wt % to 70 wt %, preferably from 50.0 wt % to 65.0 wt. %. In a preferred embodiment, the catalyst having catalytically active metal comprising Nickel (Ni) has a total pore volume in the range from 0.25 cm³/g to 0.5 cm³/g.

The remainder of the catalyst may be an oxidic bonding material, for example aluminum oxide, or zirconium dioxide. Preferably, the Ni-containing catalysts are bulk catalysts or precipitation-type catalysts, as opposed to supported catalysts.

In a preferred embodiment, the at least one catalytically active metal is Nickel (Ni).

In a preferred embodiment, the at least one catalytically active metal is Nickel-Copper (Ni—Cu).

Preferably, the molar ratio of nickel to copper is greater than 1, preferably greater than 1.2, more preferably 1.8 to 8.5.

Preferably, the at least one catalytically active metal Nickel (Ni) has Nickel content in the range from 50.0 wt % to 65.0 wt. %; and a total pore volume in the range from 0.25 cm³/g to 0.5 cm³/g.

It is well known that pure nickel catalysts tend to deposit carbon when exposed to an atmosphere containing CO or hydrocarbons (as starting materials or end products). On the other hand copper, irrespective of whether it is active or inactive as a catalyst for that reaction, does not show any tendency to deposit carbon. Therefore, nickel-copper catalysts are used for catalyzing reactions in which carbon monoxide and/or hydrocarbons are present in the reaction gas phase (as starting materials or end products) or are intermediately formed, in order to inhibit carbon deposition on the catalyst. Similarly, copper and copper alloys can be used in combination with other catalysts used for catalyzing reactions in which carbon monoxide and/or hydrocarbons are present in the reaction gas phase or are intermediately formed, in order to inhibit carbon deposition on the catalyst. In addition, presence of copper can strengthen the nickel based catalysts against attrition during catalytic reactions at high temperature and pressure conditions.

In general, precipitation methods are used for the preparation of the Ni-containing catalysts. Thus, they can be obtained, for example, by coprecipitation of the nickel and, optionally, copper components, from an aqueous salt solution containing these components, by means of a base, in the presence of a suspension of a sparingly soluble, oxidic bonding material, and subsequent washing, drying and calcination of the resulting precipitate.

For precipitation, a base, in particular an aqueous alkali metal base, for example sodium carbonate, sodium hydroxide, potassium carbonate or potassium hydroxide, is added to an aqueous salt solution containing catalyst components at elevated temperatures and with stirring, until the precipitation is complete. The type of salts used is in general not critical and salts having high aqueous solubility are generally preferred.

In a preferred embodiment, the at least one catalytically active metal comprises Nickel (Ni), and the step of contacting is carried out at a temperature in the range from 200° C. to 400° C., preferably 200 to 300° C., more preferably 200 to 250° C. When the at least one catalytically active metal is Nickel-Copper (Ni—Cu), a temperature in the range of from 250 to 350° C. is particularly useful.

In a preferred embodiment, the at least one catalyst has a particle size in the range from 50 μm to 500 μm.

In a preferred embodiment, the at least one catalyst has a particle size in the range from 100 μm to 350 μm.

In a preferred embodiment, the at least one catalyst has a bed volume in the range from 0.1 ml to 1.5 ml.

In a preferred embodiment, the at least one catalyst has a bed volume in the range from 0.2 ml to 1.1 ml.

To keep the catalyst beds on a constant level, the catalyst material can be diluted with inert particles in such way that all reactors had a constant catalyst bed volume.

In a preferred embodiment, the step of contacting involves contacting a feed stream comprising the at least one alcohol of formula (II) in a gas phase.

In a preferred embodiment, the feed stream is fed at a gas hourly space velocity (GHSV) in the range from 100 h⁻¹ to 15000 h⁻¹.

In a preferred embodiment, the feed stream is fed at a gas hourly space velocity (GHSV) in the range from 300 h⁻¹ to 10000 h⁻¹.

In a preferred embodiment, the feed stream is fed at a gas hourly space velocity (GHSV) in the range from 300 h⁻¹ to 3000 h⁻¹.

In a preferred embodiment, amount of the at least one alcohol of formula (II) in the feed stream is in the range from 2.0 wt % to 100.0 wt % based on the total weight of the feed stream.

In a preferred embodiment, amount of the at least one alcohol of formula (II) in the feed stream is in the range from 75.0 wt % to 95.0 wt % based on the total weight of the feed stream.

In a preferred embodiment, the feed stream further comprises at least one inert gas selected from N₂, CO₂, CH_{4 or Ar}.

Inert gases such as nitrogen (N₂), Carbon-di-oxide (CO₂), Argon (Ar), or methane (CH₄) can be used as diluent to adjust partial pressure of a hydrocarbon feed.

Hydrogen (H₂) gas when used as a diluent can act as an inhibitor, thereby inhibiting the coke formation on the catalyst and help improve the catalyst performance.

The skilled person will appreciate that the method is carried out in an essentially non-oxidative atmosphere, which means that the feed stream is essentially free of gaseous oxidants such as air, oxygen, ozone, nitrous oxide and nitric oxide. In particular, the feed stream is essentially free of molecular oxygen, for example, the feed stream contains less than 5 vol.-%, more preferably less than 1 vol.-% of molecular oxygen.

In a preferred embodiment, the at least one olefin of formula (I) is obtained in the form of a product stream.

In a preferred embodiment, the product stream comprises at least one olefin of formula (I), carbon monoxide and hydrogen.

In a preferred embodiment, the step of contacting further involves a step of separating carbon monoxide and hydrogen from the product stream to obtain the at least one olefin of formula (I).

The presently claimed invention is associated with at least one of the following advantages:
The presently claimed invention provides a process for conversion of an alcohol into olefins.
The presently claimed invention provides a process for consecutive dehydrogenation and dehydroformylation of renewable oxygenated biomass derivative such as an alcohol into an olefin.
The presently claimed invention provides a catalytic dehydroformylation process generating synthesis gas (gaseous CO+H₂) as a valuable by-product.
The presently claimed invention provides an efficient, economical and low temperature catalytic dehydroformylation process.
The presently claimed invention provides a catalytic dehydroformylation process involving up to 100% conversion of isoamyl alcohol.
The presently claimed invention provides a catalytic dehydroformylation process involving up to 100% conversion of butanol.
The presently claimed invention provides a catalytic dehydroformylation process involving a high carbon selectivity of 61% for propene and 35% for carbon monoxide (CO) (theoretical up to 75% and 25% respectively).

### EMBODIMENTS

In the following, there is provided a list of embodiments to further illustrate the present disclosure without intending to limit the disclosure to the specific embodiments listed below.
1. A method for preparing at least one olefin of formula (I),

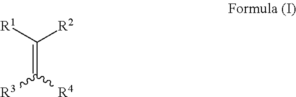

wherein, R¹, R², R³ and R⁴ are independently selected from hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, substituted C₂-C₂₀ alkynyl, C₆-C₂₀ aryl, substituted C₆-C₂₀ aryl, C₅-C₂₀ cycloalkyl, substituted C₅-C₂₀ cycloalkyl, C₇-C₂₀ arylalkyl, or substituted C₇-C₂₀ arylalkyl; or R¹ and R², together with the carbon to which they are attached, form a C₅-C₁₅ cycle,
the method comprising:
a step of contacting at least one alcohol of formula (II),

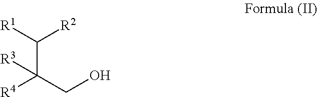

wherein, R¹, R², R³ and R⁴ are as defined above;
with at least one catalyst comprising at least one catalytically active metal selected from Platinum (Pt), Palladium (Pd), Nickel (Ni), Chromium (Cr), Molybdenum (Mo), Ruthenium (Ru), or combinations thereof, at a partial pressure in the range of 0.02 to 4.0 bar, and at a temperature in the range of 200° C. to 450° C.
2. The method according to embodiment 1, wherein R³ and R⁴ are hydrogen.
3. The method according to embodiment 1 or 2, wherein R¹ and R² are independently selected from hydrogen, C₁-C₂₀ alkyl, or substituted C₁-C₂₀ alkyl; and R³ and R⁴ are hydrogen.
4. The method according to any one of embodiments 1 to 3, wherein R¹ is C₁-C₂₀ alkyl, and R², R³ and R⁴ are hydrogen.
5. The method according to any one of embodiments 1 to 4, wherein the at least one alcohol of formula (II) is selected from propanol, butanol, isoamyl alcohol, pentanol, hexanol, heptanol, octanol, nonanol, decanol, or undecanol.
6. The method according to any one of embodiments 1 to 5, wherein the at least one catalyst comprises the at least one catalytically active metal in an amount from 0.005 wt % to 10.0 wt %, with respect to total weight of the catalyst.
7. The method according to any of embodiments 1 to 6, wherein the at least one catalyst comprises
   a. at least one catalytically active metal selected from Platinum (Pt), Palladium (Pd), Nickel (Ni), Chromium (Cr), Molybdenum (Mo), Ruthenium (Ru), or combinations thereof; and
   b. at least one promoter selected from Tin (Sn), Silver (Ag), Iridium (Ir), Rhenium (Re), or combinations thereof, wherein the at least one promoter is present in an amount in the range from 0.005 wt % to 5.0 wt %, with respect to total weight of the catalyst.
8. The method according to any of embodiments 1 to 7, wherein the at least one catalyst comprises
   a. at least one catalytically active metal selected from Platinum (Pt), Palladium (Pd), Nickel (Ni), Chromium (Cr), Molybdenum (Mo), Ruthenium (Ru), or combinations thereof; and
   b. at least one carrier on which the catalytically active metal is supported, wherein the at least one carrier is selected from a non-acidic material selected from calcium oxide, titanium oxide, beryllia, magnesia, thoria, zirconia, alumina, silicon carbide, quartz or combinations thereof, and
      wherein the at least one carrier is present in an amount in the range from 90.0 wt % to 99.995 wt %, with respect to total weight of the catalyst.
9. The method according to any of embodiments 1 to 8, wherein the at least one catalyst comprises
   a. at least one catalytically active metal selected from Platinum (Pt), Palladium (Pd), Nickel (Ni), Chromium (Cr), Molybdenum (Mo), Ruthenium (Ru), or combinations thereof;
   b. at least one promoter selected from Tin (Sn), Silver (Ag), Iridium (Ir), Rhenium (Re), or combinations thereof; and
   c. at least one carrier on which the catalytically active metal is supported, wherein the carrier is selected from a non-acidic material selected from calcium oxide, titanium oxide, beryllia, magnesia, thoria, zirconia, alumina, silicon carbide, quartz or combinations thereof.
10. The method according to embodiment 9, wherein the at least one catalytically active metal is Platinum (Pt), at least one promoter is Tin (Sn), and at least one carrier is Zirconia (ZrO₂).
11. The method according to any one of embodiments 1 to 10, wherein the at least one catalyst has a BET surface area in the range from 1 to 500 m²/g, as measured by the Brunauer-Emmett-Teller method.
12. The method according to any one of embodiments 1 to 11, wherein the at least one catalyst has a pore volume in the range from 0.1 to 1.0 ml/g, as determined by Mercury (Hg) Porosimetry.
13. The method according to any one of embodiments 1 to 12, wherein the at least one catalyst has a mean pore diameter, as determined by Mercury (Hg) Porosimetry, in the range from 0.008 to 0.06 μm.
14. The method according to any one of embodiments 1 to 13, wherein the step of contacting is carried out at a temperature in the range from 250° C. to 450° C.
15. The method according to any one of embodiments 1 to 5, wherein the at least one catalytically active metal is Palladium (Pd).
16. The method according to any one of embodiments 1 to 5, wherein the at least one catalytically active metal is Nickel (Ni).
17. The method according to any one of embodiments 15 to 16, wherein the step of contacting is carried out at a temperature in the range from 200° C. to 250° C.
18. The method according to any one of embodiments 1 to 17, wherein the at least one catalyst has a particle size in the range from 50 μm to 500 μm.
19. The method according to any one of embodiments 1 to 18, wherein the at least one catalyst has a particle size in the range from 100 μm to 350 μm.
20. The method according to any one of embodiments 1 to 19, wherein the at least one catalyst has a bed volume in the range from 0.1 ml to 1.5 ml.
21. The method according to any one of embodiments 1 to 20, wherein the at least one catalyst has a bed volume in the range from 0.2 ml to 1.1 ml.
22. The method according to any one of embodiments 1 to 21, wherein the step of contacting involves contacting a feed stream comprising the at least one alcohol of formula (II) in a gas phase.
23. The method according to any one of embodiments 1 to 22, wherein the feed stream is fed at a gas hourly space velocity (GHSV) in the range from 300 h⁻¹ to 15000 h⁻¹.
24. The method according to any one of embodiments 1 to 23, wherein the feed stream is fed at a gas hourly space velocity (GHSV) in the range from 300 h⁻¹ to 10000 h⁻¹.
25. The method according to any one of embodiments 1 to 24, wherein the feed stream is fed at a gas hourly space velocity (GHSV) in the range from 300 h⁻¹ to 3000 h⁻¹.
26. The method according to any one of embodiments 1 to 25, wherein amount of the at least one alcohol of formula (II) in the feed stream is in the range from 2.0 wt % to 100.0 wt % based on the total weight of the feed stream.
27. The method according to any one of embodiments 1 to 26, wherein amount of the at least one alcohol of formula (II) in the feed stream is in the range from 75.0 wt % to 95.0 wt % based on the total weight of the feed stream.
28. The method according to any one of embodiments 1 to 27 wherein the feed stream further comprises at least one inert gas selected from N₂, CO₂, CH₄ or Ar.
29. The method according to any of embodiments 1 to 28, wherein the at least one olefin of formula (I) is obtained in the form of a product stream.
30. The method according to any one of embodiments 1 to 29, wherein the product stream comprises at least one olefin of formula (I), carbon monoxide and hydrogen.
31. The method according to any one of embodiments 1 to 31 involves a step of separating carbon monoxide and hydrogen from the product stream to obtain the at least one olefin of formula (I).

### EXAMPLES

The presently claimed invention is illustrated in detail by non-restrictive working examples which follow. More particularly, the test methods specified hereinafter are part of the general disclosure of the application and are not restricted to the specific working examples.

### Materials and Method

Butanol pure (boiling point 117.7° C.).
Isoamyl alcohol pure (boiling point 131° C.).
Fusel alcohol (70% isoamyl alcohol+others).
Density of Pt/ZrO₂ catalyst=1.18 g/cm³.

The reactions were carried out using a highly flexible reactor (HiFlexT: R0027) equipped with 16 individual reactors, each with a heater capable of achieving temperatures up to 750° C. (850° C.), at a pressure of up to 100 bar. Each reactor had capability of holding a catalyst up to 2.0 ml.

The powdered catalysts were sieved (or screened) prior to the catalytic testing experiments. For the catalytic testing experiments a split fraction in the range of 100 to 350 μm was used.

Examples were conducted at three different reactor filling levels (or filling volumes), which were 0.25, 0.5 and 1.0 ml. The details are listed in the respective Tables. This means that the amount of catalyst which was used for an individual experiment was in the range of 0.295 to 1.18 g in case of Pt-metal on zirconium oxide (which has a density of 1.18 g/cm³). To keep the catalyst beds on a constant level, the catalyst material were diluted with inert particles in such way that all reactors had a constant filling volume.

The components of product feed/stream were detected and quantified using a Gas Chromatography system. The Gas Chromatography system was calibrated using GC standard/s for prenol, prenal, and isoprenal. The Gas Chromatography system was calibrated for products such as: CO, H₂, C₁-C₆ alkanes and alkenes, isoamyl alcohol, 3-methyl butanal, isobutene, isoprenol, and isoprene.

The following examples are included to further illustrate the invention.

A series of catalysts were used for the consecutive dehydrogenation/retro-dehydroformylation reaction in a single fixed bed reactor. The catalysts used for these reactions contained 0.005 wt % to 10.0 wt % of catalytically active metal (based on total weight of the catalyst). Optionally, the catalyst further contained sufficient amount of a promoter in an amount from 0.005 wt % to 5.0 wt %, with respect to total weight of the catalyst. The catalysts were charged to a conventional vapor phase reactor having temperature control, inlet/s for introduction of feed including inert/carrier/hydrogen gas and outlets for withdrawal of products. The reactions were carried out by varying temperatures from 200 to 450° C., and weight ratios of alcohol in the feed from 2.0 wt % to 88.0 wt % (based on total weight of the feed). The yield of the olefin for each catalyst was determined at different conversion levels.

The data is summarized in the Figures and showed that the catalyst containing a 1.0 wt % Pt/0.5 wt % Sn/ZrO₂ was unique.

n-Butanol to Propene:

TABLE 1
n-Butanol to propene using 1.0 ml of 1.0 wt % Pt/0.5 wt % Sn/ZrO₂ as catalyst
Pressure: 1.88 bar.
Product: Propene

| | Catalyst | | | Feed | | | Product (Molar ratio) | | |
|---|---|---|---|---|---|---|---|---|---|
| Exp. | Amount (in | Temp. | WHSV | Alcohol | Inert gas | Conversion | Propene/ | CO/BuOH | H₂/BuOH |
| No. | Volume) | ° C. | h⁻¹ | (wt %) | (wt %) | [%] | BuOH (in) | (in) | (in) |
| 1. | 1.0 ml | 400 | 1.5 | n-Butanol | N₂ | 99.1 | 0.51 | 0.63 | 1.33 |
| | | | | (9 wt %) | (86 wt %) | | | | |

From Table 1, it is evident that by using 1.0 ml of 1.0 wt % Pt/0.5 wt % Sn/ZrO₂ as catalyst up to 100% conversion of butanol was achieved.

Thus, a carbon selectivity of 61.0% for propene and 35.0% for CO was achieved (theoretical up to 75% and 25% respectively).

### Isoamyl Alcohol to Isobutene:

TABLE 2
Conversion of isoamyl alcohol using 0.25 ml of 1.0 wt % Pt/0.5 wt % Sn/ZrO₂ as catalyst

| | | | | Gas | Gas | Isoamyl | | |
|---|---|---|---|---|---|---|---|---|
| Experiment | CatalystAmount | Temperature | Pressure | N₂ | Ar | Alcohol | GHSV | Conversion |
| No. | (in Volume) | ° C. | [bar] | [%] | [%] | (vol %) | [h⁻¹] | [%] |
| 2 | 0.25 ml | 200 | 1.0039975 | 80 | 10 | 9.9 | 9970 | 4.2954682 |
| 3 | 0.25 ml | 250 | 1.0039975 | 80 | 10 | 9.9 | 9970 | 12.47727 |
| 4 | 0.25 ml | 225 | 1.0039975 | 80 | 10 | 9.9 | 9970 | 6.4742367 |
| 5 | 0.25 ml | 200 | 1.0039975 | 80 | 10 | 9.9 | 9970 | 10.57326 |
| 6 | 0.25 ml | 300 | 1.0039975 | 80 | 10 | 9.9 | 9970 | 55.451812 |
| 7 | 0.25 ml | 325 | 1.0039975 | 80 | 10 | 9.9 | 9970 | 81.027303 |
| 8 | 0.25 ml | 300 | 1.0039975 | 80 | 10 | 9.9 | 9970 | 56.946055 |
| 9 | 0.25 ml | 300 | 1.0039975 | 80 | 10 | 9.9 | 9970 | 53.816486 |
| 10 | 0.25 ml | 300 | 1.0039975 | 80 | 10 | 9.9 | 9970 | 57.162374 |

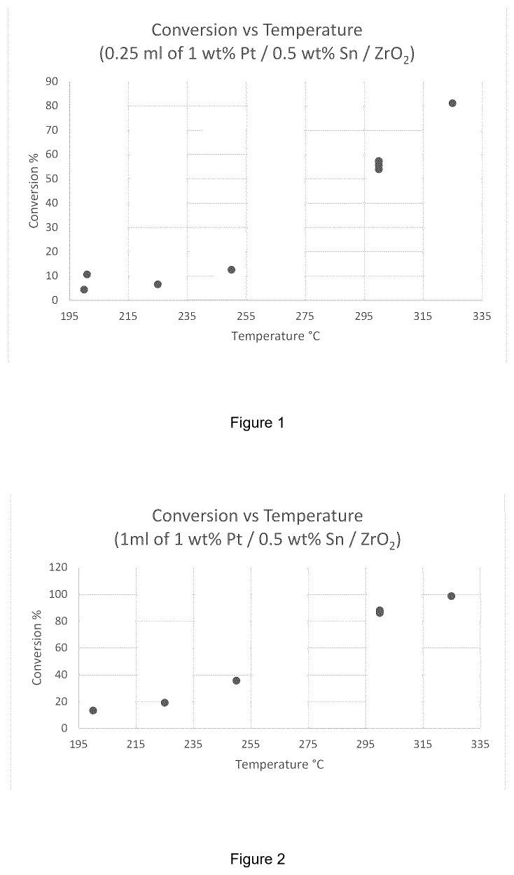

From Table 2, it is evident that on passing a gas feed of 9.9 vol % isoamyl alcohol at a GHSV of 9970 h⁻¹ over 0.25 ml of 1.0 wt % Pt/0.5 wt % Sn/ZrO₂ catalyst, 4.0% to 81.0% conversion of isoamyl alcohol was achieved at a pressure of 1.0 bar, and at a temperature in the range of 200 to 325° C. It is also evident that highest conversion (81.0%) was achieved at a temperature of 325° C. The results of Table 2 are illustrated graphically in FIG. 1.

TABLE 3
Conversion of isoamyl alcohol using 1.0 ml of 1.0 wt % Pt/0.5 wt % Sn/ZrO₂ as catalyst

| | | | | Gas | Gas | Isoamyl | | |
|---|---|---|---|---|---|---|---|---|
| Experiment | Catalyst Amount | Temperature | Pressure | N₂ | Ar | Alcohol | GHSV | Conversion |
| No. | (in Volume) | ° C. | [bar] | [%] | [%] | (vol %) | [h⁻¹] | [%] |
| 11 | 1.0 ml | 200 | 0.9903696 | 80 | 10 | 9.9 | 2500 | 3.800625 |
| 12 | 1.0 ml | 200 | 1.0039975 | 80 | 10 | 9.9 | 2500 | 13.200963 |
| 13 | 1.0 ml | 225 | 1.0039975 | 80 | 10 | 9.9 | 2500 | 18.971075 |
| 14 | 1.0 ml | 250 | 1.0039975 | 80 | 10 | 9.9 | 2500 | 35.428908 |
| 15 | 1.0 ml | 300 | 1.0039975 | 80 | 10 | 9.9 | 2500 | 86.130414 |
| 16 | 1.0 ml | 300 | 0.9940639 | 80 | 10 | 9.9 | 2500 | 87.284889 |
| 17 | 1.0 ml | 300 | 1.0039975 | 80 | 10 | 9.9 | 2500 | 85.842945 |
| 18 | 1.0 ml | 300 | 1.0039975 | 80 | 10 | 9.9 | 2500 | 87.874541 |
| 19 | 1.0 ml | 325 | 1.0039975 | 80 | 10 | 9.9 | 2500 | 98.393322 |

From Table 3, it is evident that on passing a gas feed of 9.9 vol % isoamyl alcohol at a GHSV of 2500 h¹ over 1.0 ml of 1.0 wt % Pt/0.5 wt % Sn/ZrO₂ catalyst, 3.8 to 98.4% conversion of isoamyl alcohol was achieved at a pressure of 1.0 bar, and at a temperature in the range of 200 to 325° C. It is also evident that highest conversion (98.4%) was achieved at a temperature of 325°

TABLE 4
Conversion of isoamyl alcohol using 1.0 ml of 1.0 wt % Pt/0.5
wt % Sn/ZrO₂ as catalyst at a constant GHSV of 2500 h⁻¹.

| | | | | Gas | Gas | Isoamyl | | |
|---|---|---|---|---|---|---|---|---|
| Experiment | Catalyst Amount | Temperature | Pressure | N₂ | Ar | Alcohol | GHSV | Conversion |
| No. | (in Volume) | ° C. | [bar] | [%] | [%] | (vol %) | [h⁻¹] | [%] |
| 20 | 1.0 ml | 275 | 1.0039975 | 80 | 10 | 9.9 | 2500 | 70.575313 |
| 21 | 1.0 ml | 300 | 0.9940639 | 80 | 10 | 9.9 | 2500 | 93.032982 |
| 22 | 1.0 ml | 300 | 1.0039975 | 80 | 10 | 9.9 | 2500 | 88.886833 |
| 23 | 1.0 ml | 300 | 1.0039975 | 80 | 10 | 9.9 | 2500 | 89.507002 |
| 24 | 1.0 ml | 300 | 1.0039975 | 80 | 10 | 9.9 | 2500 | 88.492228 |
| 25 | 1.0 ml | 300 | 1.0039975 | 80 | 10 | 9.9 | 2500 | 91.738112 |
| 26 | 1.0 ml | 325 | 1.0039975 | 80 | 10 | 9.9 | 2500 | 97.193903 |
| 27 | 1.0 ml | 325 | 1.0039975 | 80 | 10 | 9.9 | 2500 | 97.169145 |
| 28 | 1.0 ml | 350 | 1.0039975 | 80 | 10 | 9.9 | 2500 | 100 |

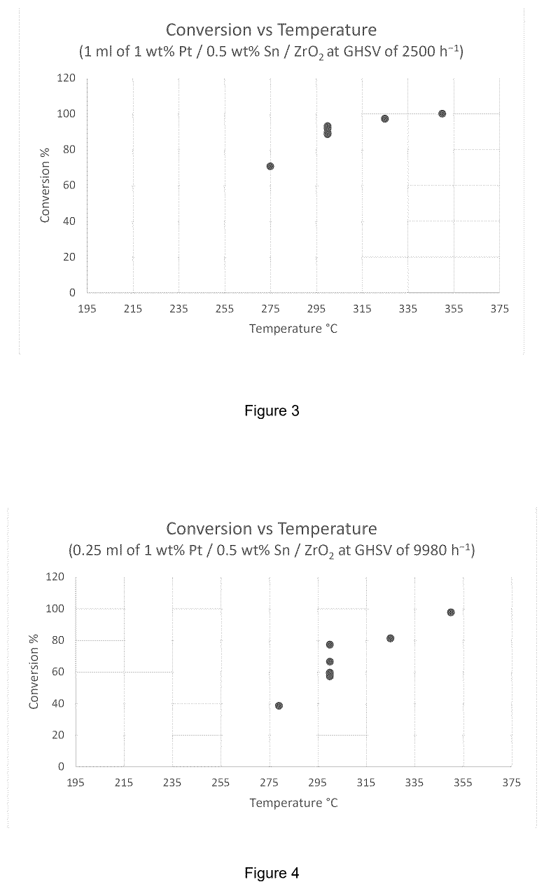

From Table 4, it is evident that on passing a gas feed of 9.9 vol % isoamyl alcohol at a GHSV of 2500 h⁻¹ over 1.0 ml of 1.0 wt % Pt/0.5 wt % Sn/ZrO₂ catalyst, 70.6% to 100.0% conversion of isoamyl alcohol was achieved at a pressure of 1.0 bar, and at a temperature in the range of 275 to 350° C. It is also evident that highest conversion (100.0%) was achieved at a temperature of 350° C. The results of Table 4 are presented graphically in FIG. 3. From Experiments 11 to 28, it was observed that the yield of the isobutene was in the range of 1 mol % to 99 mol %.

It is evident from Tables 2-4 that the conversion of Isoamyl alcohol increased with an increase in the amount of the catalyst from 0.25 ml to 1.0 ml, and with a decrease in GHSV of 9.9 vol % isoamyl alcohol from 9970 h⁻¹ to 2500 h⁻¹.

It is evident from Tables 2-4 that the conversion of Isoamyl alcohol in the presence of 1.0 ml of 1.0 wt % Pt/0.5 wt % Sn/ZrO₂ as catalyst, increased with the temperature in the range of 250° C. to 350° C. Thus, conversion of Isoamyl alcohol in the presence of the 1.0 wt % Pt/0.5 wt % Sn/ZrO₂ as catalyst was achieved in the range of 250° C. to 450° C.

TABLE 5
Conversion of Isoamyl alcohol using 0.25 ml of 1.0 wt % Pt/0.5
wt % Sn/ZrO₂ as catalyst at a constant GHSV of 9800 h⁻¹.

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 29 | 0.25 ml | 275 | 1.0039975 | 80 | 10 | 9.9 | 9980 | 38.478529 |
| 30 | 0.25 ml | 300 | 1.0039975 | 80 | 10 | 9.9 | 9980 | 77.02082 |
| 31 | 0.25 ml | 300 | 1.0039975 | 80 | 10 | 9.9 | 9980 | 57.067727 |
| 32 | 0.25 ml | 300 | 1.0039975 | 80 | 10 | 9.9 | 9980 | 59.438213 |
| 33 | 0.25 ml | 300 | 1.0039975 | 80 | 10 | 9.9 | 9980 | 58.604101 |
| 34 | 0.25 ml | 300 | 1.0039975 | 80 | 10 | 9.9 | 9980 | 66.349825 |
| 35 | 0.25 ml | 325 | 1.0039975 | 80 | 10 | 9.9 | 9980 | 81.028797 |
| 36 | 0.25 ml | 325 | 1.0039975 | 80 | 10 | 9.9 | 9980 | 81.161481 |
| 37 | 0.25 ml | 350 | 1.0039975 | 80 | 10 | 9.9 | 9980 | 97.435403 |

From Table 5, it is evident that on passing a gas feed of 9.9 vol % isoamyl alcohol at a GHSV of 9980 h⁻¹ over 0.25 ml of 1.0 wt % Pt/0.5 wt % Sn/ZrO₂ catalyst, 38.5% to 97.4% conversion of isoamyl alcohol was achieved at a pressure of 1.0 bar, and at a temperature in the range of 275 to 350° C. It is also evident that highest conversion (97.4%) was achieved at a temperature of 350° C. The results of Table 5 are presented graphically in FIG. 4.

TABLE 6
Conversion of Isoamyl alcohol using 1.0 ml of 1.0 wt % Pt/0.5 wt %
Sn/ZrO₂ as catalyst at various GHSV's, at Temp = 300, and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 38 | 1.0 ml | 300 | 0.9940639 | 80 | 10 | 9.9 | 2500 | 93.032982 |
| 39 | 1.0 ml | 300 | 3.5232452 | 0 | 20 | 80.1 | 320 | 100 |
| 40 | 1.0 ml | 300 | 1.0751738 | 0 | 20 | 80 | 2500 | 47.993518 |
| 41 | 1.0 ml | 300 | 1.0039975 | 0 | 20 | 80.1 | 320 | 77.846418 |
| 42 | 1.0 ml | 300 | 1.0039975 | 50 | 10 | 40.1 | 640 | 81.782255 |
| 43 | 1.0 ml | 300 | 1.0039975 | 80 | 10 | 9.9 | 2500 | 88.886833 |
| 44 | 1.0 ml | 300 | 1.0039975 | 80 | 10 | 9.9 | 2500 | 89.507002 |
| 45 | 1.0 ml | 300 | 1.0039975 | 80 | 10 | 9.9 | 2500 | 88.492228 |
| 46 | 1.0 ml | 300 | 1.0039975 | 88 | 10 | 2 | 2500 | 99.21187 |
| 47 | 1.0 ml | 300 | 1.0039975 | 80 | 10 | 9.9 | 2500 | 91.738112 |

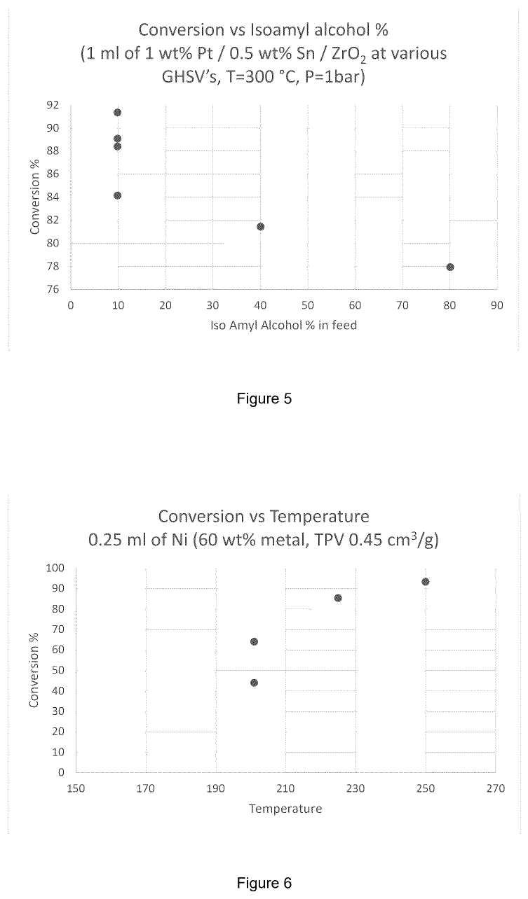

From Table 6, it is evident that on passing a gas feed of 2.0 vol % to 80.1 vol % isoamyl alcohol at a GHSV in the range of 320 h⁻¹ to 2500 h⁻¹, over 0.25 ml of 1.0 wt % Pt/0.5 wt % Sn/ZrO₂ catalyst, at a pressure of 1.0 bar, and at a temperature of 300° C., 47.9% to 100.000 conversion of isoamyl alcohol was achieved. The results of Table 6 are presented graphically in FIG. 5.

It is evident from Tables 4-6 that the conversion of Isoamyl alcohol decreased with the increasing GHSV. This could be because of the reduction in contact of the isoamyl alcohol in the feed with the catalyst due to increased velocity. Thus, the conversion was optimised by adjusting the amount of isoamyl alcohol in the feed with the change in GHSV in order to increase the contact between isoamyl alcohol in the feed and the catalyst. The conversion of Isoamyl alcohol is highest when the GHSV of the isoamyl alcohol in the feed is in the range of 320 h⁻¹ to 2500 h⁻¹ and the amount of isoamyl alcohol in the feed is in the range of 2.0 vol % to 80.1 vol %.

TABLE 7
Conversion of Isoamyl alcohol using 0.25 ml of Nickel (60 wt % metal content, Total
Pore Volume 0.45 cm³/g) as catalyst at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 48 | 0.25 ml | 200 | 1.0039975 | 80 | 10 | 9.9 | 9920 | 63.901511 |
| 49 | 0.25 ml | 200 | 1.0039975 | 80 | 10 | 9.9 | 9920 | 43.804172 |
| 50 | 0.25 ml | 225 | 1.0039975 | 80 | 10 | 9.9 | 9920 | 85.155518 |
| 51 | 0.25 ml | 250 | 1.0039975 | 80 | 10 | 9.9 | 9920 | 93.147337 |

From Table 7, it is evident that on passing a gas feed of 9.9 vol % isoamyl alcohol at a GHSV of 9920 h⁻¹ over 0.25 ml of Nickel (60 wt % metal content, Total Pore Volume 0.45 cm³/g), 43.8% to 93.1% conversion of isoamyl alcohol was achieved at a pressure of 1.0 bar, and at a temperature in the range of 200 to 250° C. It is also evident that highest conversion (93.1%) was achieved at a temperature of 250° C. The results of Table 7 are presented graphically in FIG. 6.

It is evident from Table 7 and FIG. 6, that 0.25 ml of Nickel (60 wt % metal content, Total Pore Volume 0.45 cm³/g) as catalyst is actively involved in 43% to 94% conversion of isoamyl alcohol.

TABLE 8
Conversion of Isoamyl alcohol using 1.0 ml of Nickel (60 wt % metal content, Total
Pore Volume 0.45 cm³/g) as catalyst at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 52 | 1.0 ml | 200 | 1.0039975 | 80 | 10 | 9.9 | 2500 | 80.341941 |
| 53 | 1.0 ml | 200 | 1.0039975 | 80 | 10 | 9.9 | 2500 | 61.865841 |
| 54 | 1.0 ml | 225 | 1.0039975 | 80 | 10 | 9.9 | 2500 | 84.345723 |
| 55 | 1.0 ml | 230 | 1.0039975 | 80 | 10 | 9.9 | 2500 | 89.495523 |
| 56 | 1.0 ml | 239 | 1.0039975 | 80 | 10 | 9.9 | 2500 | 90.918375 |
| 57 | 1.0 ml | 239 | 1.0039975 | 80 | 10 | 9.9 | 2500 | 86.910073 |
| 58 | 1.0 ml | 239 | 1.0039975 | 80 | 10 | 9.9 | 2500 | 74.166114 |
| 59 | 1.0 ml | 250 | 1.0039975 | 80 | 10 | 9.9 | 2500 | 92.431857 |

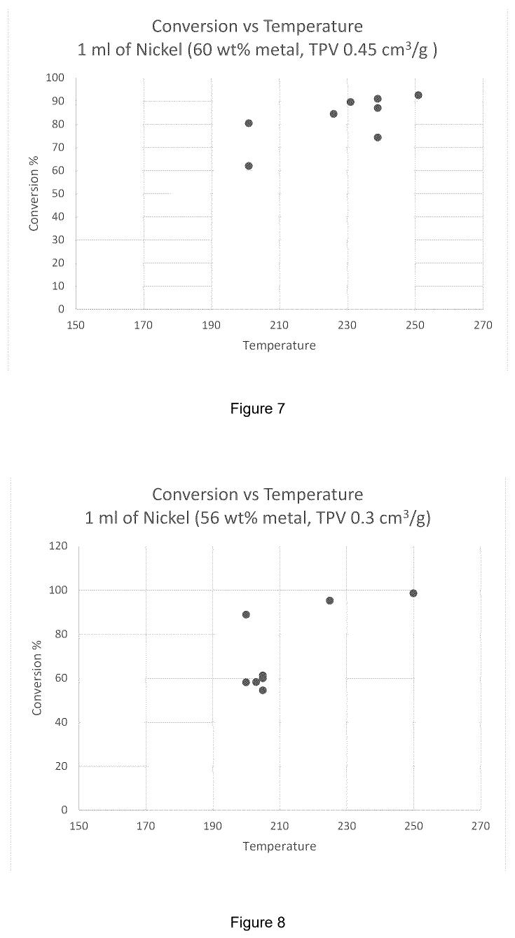

From Table 8, it is evident that on passing a gas feed of 9.9 vol % isoamyl alcohol at a GHSV of 2500 h⁻¹ over 1.0 ml of Nickel (60 wt % metal content, Total Pore Volume 0.45 cm³/g), 61.8% to 92.4% conversion of isoamyl alcohol was achieved at a pressure of 1.0 bar, and at a temperature above 200° C. It is also evident that highest conversion (92.4%) was achieved at a temperature of 250° C. The results of Table 8 are presented graphically in FIG. 7. From Experiments 52 to 59, it was observed that the yield of the isobutene was in the range of 1 mol % to 99 mol %.

It is evident from Table 8 and FIG. 7, that 1.0 ml of Nickel (60 wt % metal content, Total Pore Volume 0.45 cm³/g) as catalyst is actively involved in 61% to 93% conversion of isoamyl alcohol.

TABLE 9
Conversion of Isoamyl alcohol using 1.0 ml of Nickel (56 wt % metal content, Total
Pore Volume 0.30 cm³/g) as catalyst at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 60 | 1.0 ml | 200 | 1.0039975 | 80 | 10 | 9.9 | 2500 | 88.768356 |
| 61 | 1.0 ml | 200 | 1.0039975 | 80 | 10 | 9.9 | 2500 | 57.976593 |
| 62 | 1.0 ml | 200 | 1.0039975 | 80 | 10 | 9.9 | 2500 | 58.091771 |
| 63 | 1.0 ml | 205 | 1.0039975 | 80 | 10 | 9.9 | 2500 | 61.139635 |
| 64 | 1.0 ml | 205 | 1.0039975 | 80 | 10 | 9.9 | 2500 | 59.875445 |
| 65 | 1.0 ml | 205 | 1.0039975 | 80 | 10 | 9.9 | 2500 | 54.408384 |
| 66 | 1.0 ml | 225 | 1.0039975 | 80 | 10 | 9.9 | 2500 | 95.125146 |
| 67 | 1.0 ml | 250 | 1.0039975 | 80 | 10 | 9.9 | 2500 | 98.450192 |

From Table 9, it is evident that on passing a gas feed of 9.9 vol % isoamyl alcohol at a GHSV of 2500 h⁻¹ over 1.0 ml of Nickel (56 wt % metal content, Total Pore Volume 0.3 cm³/g), 54.4% to 98.4% conversion of isoamyl alcohol was achieved at a pressure of 1.0 bar, and at a temperature in the range of 200 to 250° C. It is also evident that highest conversion (98.4%) was achieved at a temperature of 250° C. The results of Table 9 are presented graphically in FIG. 8. From Experiments 60 to 67, it was observed that the yield of the isobutene was in the range of 1 mol % to 99 mol %.

It is evident from Table 9 and FIG. 8, that although 1.0 ml of Ni (56 wt % metal content, Total Pore Volume 0.3 cm³/g) as catalyst is actively involved in 54% to 98.5% conversion of isoamyl alcohol.

TABLE 10
Conversion of Isoamyl alcohol using 1.0 ml Nickel-Copper
as catalyst at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 68 | 1.0 ml | 275 | 1.0039975 | 80 | 10 | 9.9 | 2500 | 39.350433 |
| 69 | 1.0 ml | 300 | 1.0039975 | 80 | 10 | 9.9 | 2500 | 100 |
| 70 | 1.0 ml | 325 | 1.0039975 | 80 | 10 | 9.9 | 2500 | 71.233996 |
| 71 | 1.0 ml | 350 | 1.0039975 | 80 | 10 | 9.9 | 2500 | 100 |

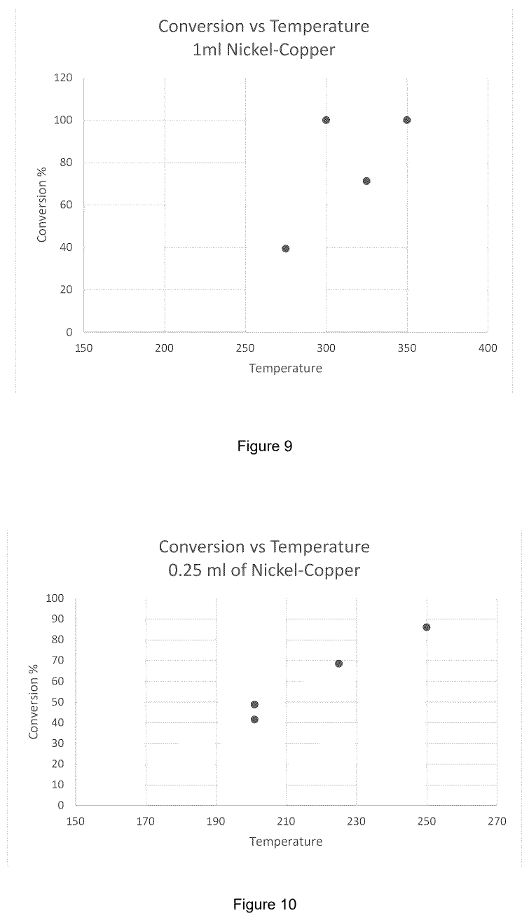

From Table 10, it is evident that on passing a gas feed of 9.9 vol % isoamyl alcohol at a GHSV of 2500 h⁻¹ over 1.0 ml of Nickel-Copper as catalyst, 39.3% to 100.0% conversion of isoamyl alcohol was achieved at a pressure of 1.0 bar, and at a temperature in the range of 275 to 350° C. It is also evident that highest conversion (100.0%) was achieved at a temperature above 300° C. The results of Table 10 are presented graphically in FIG. 9. From Experiments 68 to 71, it was observed that the yield of the isobutene was in the range of 1 mol % to 99 mol %.

It is evident from Table 10 and FIG. 9, that Nickel-Copper as catalyst is actively involved in 39% to 100% conversion of isoamyl alcohol.

TABLE 11
Conversion of Isoamyl alcohol using 0.25 ml of Nickel-Copper
as catalyst at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 72 | 0.25 ml | 200 | 1.0039975 | 80 | 10 | 9.9 | 10010 | 48.620368 |
| 73 | 0.25 ml | 200 | 1.0039975 | 80 | 10 | 9.9 | 10010 | 41.426903 |
| 74 | 0.25 ml | 225 | 1.0039975 | 80 | 10 | 9.9 | 10010 | 68.354063 |
| 75 | 0.25 ml | 250 | 1.0039975 | 80 | 10 | 9.9 | 10010 | 85.928005 |

From Table 11, it is evident that on passing a gas feed of 9.9 vol % isoamyl alcohol at a GHSV of 10010 h⁻¹ over 0.25 ml of Nickel-Copper as catalyst, 41.4% to 85.9% conversion of isoamyl alcohol was achieved at a pressure of 1.0 bar, and at a temperature in the range of 200 to 250° C. It is also evident that highest conversion (85.9%) was achieved at temperature 250° C. The results of Table 11 are presented graphically in FIG. 10.

It is evident from Table 11 and FIG. 10 that 0.25 ml of Nickel-Copper as catalyst is actively involved in 41% to 86% conversion of isoamyl alcohol.

TABLE 12
Isoamyl alcohol to isobutene using 1.0 ml of Nickel-Copper
as catalyst at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 76 | 1.0 ml | 200 | 1.0039975 | 80 | 10 | 9.9 | 2500 | 57.395141 |
| 77 | 1.0 ml | 200 | 1.0039975 | 80 | 10 | 9.9 | 2500 | 50.526326 |
| 78 | 1.0 ml | 225 | 1.0039975 | 80 | 10 | 9.9 | 2500 | 85.652415 |
| 79 | 1.0 ml | 225 | 1.0039975 | 80 | 10 | 9.9 | 2500 | 74.400105 |
| 80 | 1.0 ml | 230 | 1.0039975 | 80 | 10 | 9.9 | 2500 | 70.360868 |
| 81 | 1.0 ml | 230 | 1.0039975 | 80 | 10 | 9.9 | 2500 | 69.532824 |
| 82 | 1.0 ml | 230 | 1.0039975 | 80 | 10 | 9.9 | 2500 | 68.137744 |
| 83 | 1.0 ml | 250 | 1.0039975 | 80 | 10 | 9.9 | 2500 | 97.549106 |
| 84 | 1.0 ml | 250 | 1.0039975 | 80 | 10 | 9.9 | 2500 | 95.060903 |

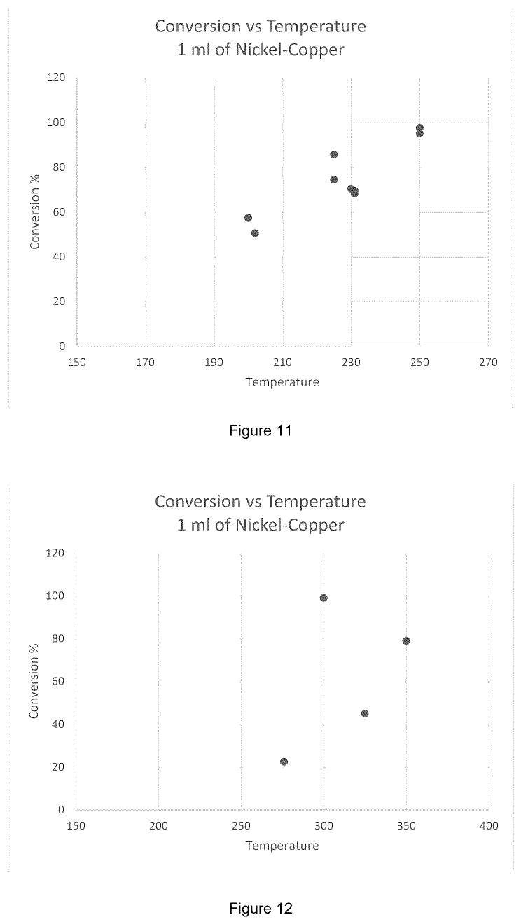

From Table 12, it is evident that on passing a gas feed of 9.9 vol % isoamyl alcohol at a GHSV of 2500 h⁻¹ over 1.0 ml of Nickel-Copper as catalyst, 50.5% to 97.5% conversion of isoamyl alcohol was achieved at a pressure of 1.0 bar, and at a temperature in the range of 200 to 250° C. It is also evident that highest conversion (97.5%) was achieved at temperature 250° C. The results of Table 12 are presented graphically in FIG. 11. From Experiments 76 to 84, it was observed that the yield of the isobutene was in the range of 1 mol % to 99 mol %.

It is evident from Table 12 and FIG. 11, that 1.0 ml of Nickel-Copper as catalyst is actively involved in 50% to 98% conversion of isoamyl alcohol.

TABLE 13
Isoamyl alcohol to isobutene using 1.0 ml Nickel-Copper
as catalyst at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 85 | 1.0 ml | 275 | 1.0039975 | 80 | 10 | 9.9 | 2500 | 22.36356 |
| 86 | 1.0 ml | 300 | 1.0039975 | 80 | 10 | 9.9 | 2500 | 98.945455 |
| 87 | 1.0 ml | 325 | 1.0039975 | 80 | 10 | 9.9 | 2500 | 44.900714 |
| 88 | 1.0 ml | 350 | 1.0039975 | 80 | 10 | 9.9 | 2500 | 78.845617 |

From Table 13, it is evident that on passing a gas feed of 9.9 vol % isoamyl alcohol at a GHSV of 2500 h⁻¹ over 1.0 ml of Nickel-Copper as catalyst, 22.3% to 98.9% conversion of isoamyl alcohol was achieved at a pressure of 1.0 bar, and at a temperature in the range of 275 to 350° C. It is also evident that highest conversion (98.9%) was achieved at temperature 300° C. The results of Table 13 are presented graphically in FIG. 12. From Experiments 85 to 88, it was observed that the yield of the isobutene was in the range of 1 mol % to 99 mol %.

It is evident from Table 13 and FIG. 12 that Nickel-Copper as catalyst is actively involved in 22% to 99% conversion of isoamyl alcohol.

TABLE 14
Isoamyl alcohol to isobutene using 0.5 ml of [1.0 wt % Pt/0.5 wt %
Sn/ZrO₂ + Cu—Zn] as catalyst at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 89 | 0.5 ml | 200 | 1.0039975 | 80 | 10 | 9.9 | 5200 | 35.28412 |
| 90 | 0.5 ml | 200 | 1.0039975 | 80 | 10 | 9.9 | 5200 | 40.866523 |
| 91 | 0.5 ml | 225 | 1.0039975 | 80 | 10 | 9.9 | 5200 | 52.356107 |
| 92 | 0.5 ml | 250 | 1.0039975 | 80 | 10 | 9.9 | 5200 | 68.565915 |
| 93 | 0.5 ml | 300 | 1.0039975 | 80 | 10 | 9.9 | 5200 | 88.979086 |
| 94 | 0.5 ml | 300 | 1.0039975 | 80 | 10 | 9.9 | 5200 | 88.675742 |
| 95 | 0.5 ml | 300 | 1.0039975 | 80 | 10 | 9.9 | 5200 | 84.173945 |
| 96 | 0.5 ml | 300 | 1.0071991 | 80 | 10 | 9.9 | 5200 | 83.172232 |
| 97 | 0.5 ml | 325 | 1.0039975 | 80 | 10 | 9.9 | 5200 | 95.451202 |

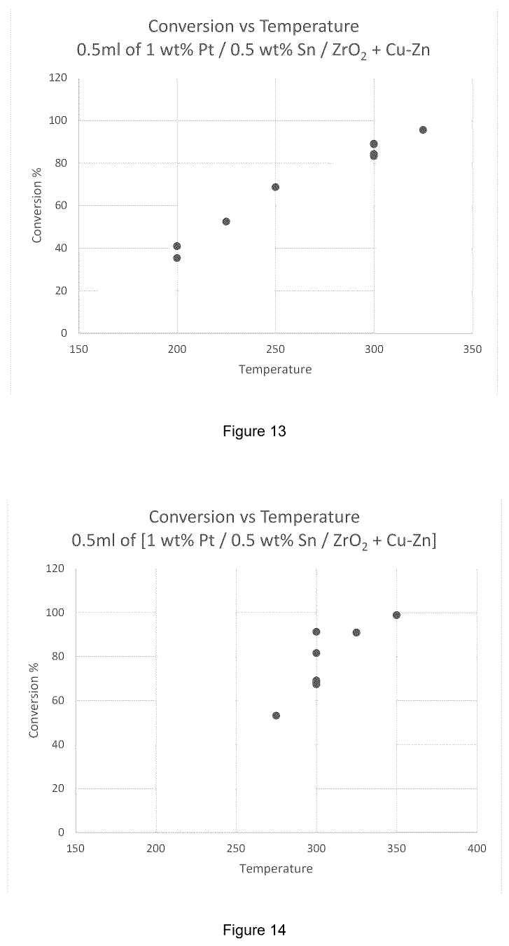

From Table 14, it is evident that on passing a gas feed of 9.9 vol % isoamyl alcohol at a GHSV of 5200 h⁻¹ over 0.5 ml of [1.0 wt % Pt/0.5 wt % Sn/ZrO₂+Cu-Zn] as catalyst, 35.3% to 95.4% conversion of isoamyl alcohol was achieved at a pressure of 1.0 bar, and at a temperature in the range of 200 to 325° C. It is also evident that highest conversion (95.4%) was achieved at temperature 325° C. The results of Table 14 are presented graphically in FIG. 13.

It is evident from Table 14 and FIG. 13, that 0.5 ml of [1.0 wt % Pt/0.5 wt % Sn/ZrO₂+Cu—Zn] as catalyst is actively involved in 35% to 96% conversion of isoamyl alcohol.

TABLE 15
Isoamyl alcohol to isobutene using 0.5 ml [1.0 wt % Pt/0.5 wt %
Sn/ZrO₂ + Cu—Zn] as catalyst at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 98 | 0.5 ml | 275 | 1.0039975 | 80 | 10 | 9.9 | 5890 | 53.045836 |
| 99 | 0.5 ml | 300 | 1.0039975 | 80 | 10 | 9.9 | 5890 | 81.500389 |
| 100 | 0.5 ml | 300 | 1.0039975 | 80 | 10 | 9.9 | 5890 | 68.141897 |
| 101 | 0.5 ml | 300 | 1.0039975 | 80 | 10 | 9.9 | 5890 | 69.026573 |
| 102 | 0.5 ml | 300 | 1.0039975 | 80 | 10 | 9.9 | 5890 | 67.251688 |
| 103 | 0.5 ml | 300 | 1.0039975 | 80 | 10 | 9.9 | 5890 | 91.153073 |
| 104 | 0.5 ml | 325 | 1.0039975 | 80 | 10 | 9.9 | 5890 | 90.719876 |
| 105 | 0.5 ml | 325 | 1.0039975 | 80 | 10 | 9.9 | 5890 | 90.808013 |
| 106 | 0.5 ml | 350 | 1.0039975 | 80 | 10 | 9.9 | 5890 | 98.715844 |

From Table 15, it is evident that on passing a gas feed of 9.9 vol % isoamyl alcohol at a GHSV of 5890 h⁻¹ over 0.5 ml of [1.0 wt % Pt/0.5 wt % Sn/ZrO₂+Cu—Zn] as catalyst, 53.0% to 98.7% conversion of isoamyl alcohol was achieved at a pressure of 1.0 bar, and at a temperature in the range of 275 to 350° C. It is also evident that highest conversion (98.7%) was achieved at temperature 350° C. The results of Table 15 are presented graphically in FIG. 14.

It is evident from Table 15 and FIG. 14 that 1.0 wt % Pt/0.5 wt % Sn/ZrO₂+Cu-Zn as catalyst is actively involved in 53% to 99% conversion of isoamyl alcohol.

TABLE 16
Isoamyl alcohol to isobutene using 1.0 ml Ru as catalyst
at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 107 | 1.0 ml | 275 | 1.0039975 | 80 | 10 | 9.9 | 2500 | 16.159157 |
| 108 | 1.0 ml | 300 | 1.0039975 | 80 | 10 | 9.9 | 2500 | 54.449142 |
| 109 | 1.0 ml | 325 | 1.0039975 | 80 | 10 | 9.9 | 2500 | 46.895054 |
| 110 | 1.0 ml | 350 | 1.0039975 | 80 | 10 | 9.9 | 2500 | 73.78532 |

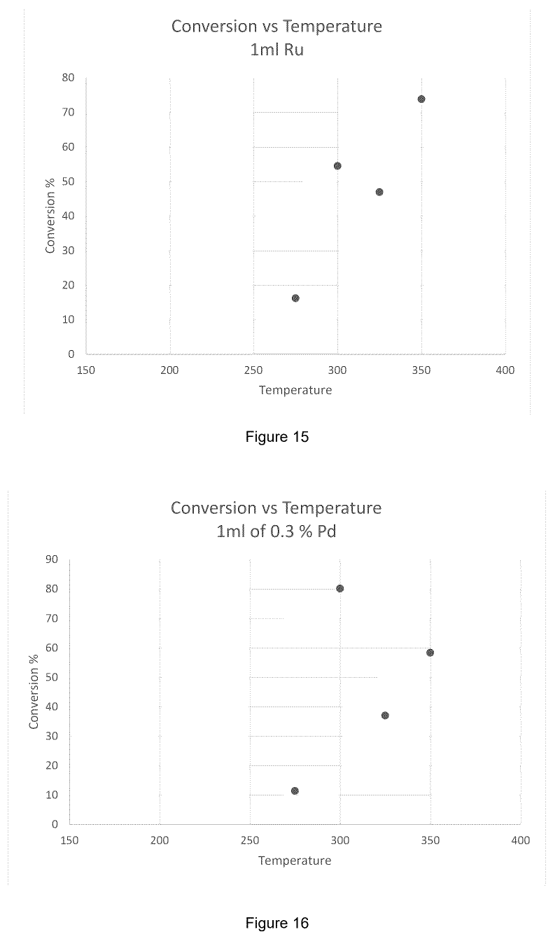

From Table 16, it is evident that on passing a gas feed of 9.9 vol % isoamyl alcohol at a GHSV of 2500 h⁻¹ over 1.0 ml of Ru as catalyst, 16.1% to 73.8% conversion of isoamyl alcohol was achieved at a pressure of 1.0 bar, and at a temperature in the range of 275 to 350° C. It is also evident that highest conversion (93.8%) was achieved at temperature 350° C. The results of Table 16 are presented graphically in FIG. 15.

It is evident from Table 16 and FIG. 15, that 1.0 ml Ru as catalyst is actively involved in 16% to 74% conversion of isoamyl alcohol.

TABLE 17
Isoamyl alcohol to isobutene using (0.3 wt % Pd) as
catalyst at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 111 | 1.0 ml | 275 | 1.0039975 | 80 | 10 | 9.9 | 2490 | 11.347244 |
| 112 | 1.0 ml | 300 | 1.0039975 | 80 | 10 | 9.9 | 2490 | 80.080656 |
| 113 | 1.0 ml | 325 | 1.0039975 | 80 | 10 | 9.9 | 2490 | 36.961334 |
| 114 | 1.0 ml | 350 | 1.0039975 | 80 | 10 | 9.9 | 2490 | 58.26616 |

From Table 17, it is evident that on passing a gas feed of 9.9 vol % isoamyl alcohol at a GHSV of 2490 h⁻¹ over 1.0 ml of 0.3% Pd as catalyst, 11.3% to 80.0% conversion of isoamyl alcohol was achieved at a pressure of 1.0 bar, and at a temperature in the range of 275 to 350° C. It is also evident that highest conversion (80.0%) was achieved at temperature 300° C. The results of Table 20 are presented graphically in FIG. 16.

It is evident from Table 20 and FIG. 16 that (0.3% Pd) as catalyst is actively involved in 11% to 81% conversion of isoamyl alcohol.

TABLE 18
Isoamyl alcohol to isobutene using 1.0 ml of (0.5 wt % Pd)
as catalyst at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 115 | 1.0 ml | 275 | 1.0039975 | 80 | 10 | 9.9 | 2500 | 45.086682 |
| 116 | 1.0 ml | 300 | 1.0039975 | 80 | 10 | 9.9 | 2500 | 97.116832 |
| 117 | 1.0 ml | 325 | 1.0039975 | 80 | 10 | 9.9 | 2500 | 86.728951 |
| 118 | 1.0 ml | 350 | 1.0039975 | 80 | 10 | 9.9 | 2500 | 95.177062 |

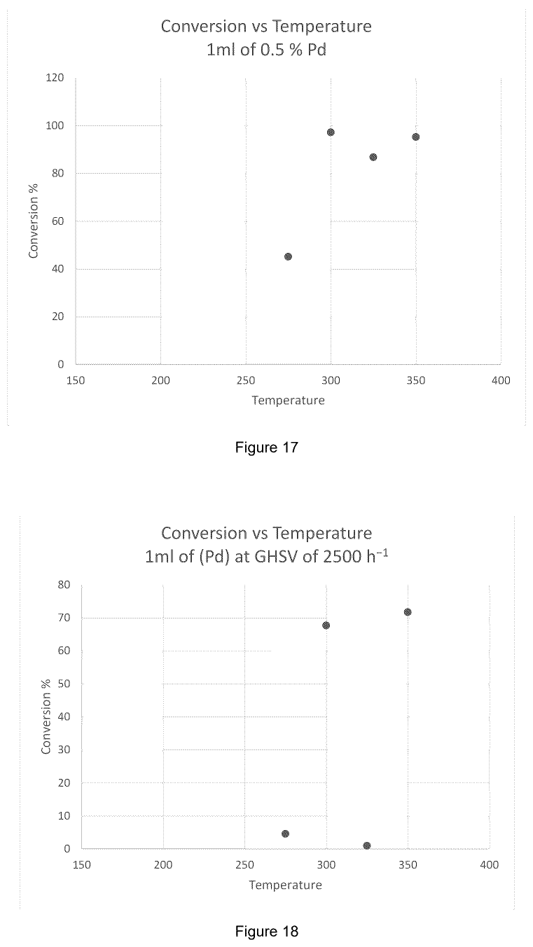

From Table 18, it is evident that on passing a gas feed of 9.9 vol % isoamyl alcohol at a GHSV of 2500 h⁻¹ over 1.0 ml of 0.5% Pd as catalyst, 45.1% to 97.1% conversion of isoamyl alcohol was achieved at a pressure of 1.0 bar, and at a temperature in the range of 275 to 350° C. It is also evident that highest conversion (97.1%) was achieved at temperature 300° C. The results of Table 18 are presented graphically in FIG. 17.

It is evident from Table 18 and FIG. 17, that (0.5% Pd) as catalyst is actively involved in 45% to 97% conversion of isoamyl alcohol.

TABLE 19
Isoamyl alcohol to isobutene using 1 ml of (Pd) as catalyst
at various temperatures and Pressure = 1 bar

| | Catalyst | | | | | | | |
|---|---|---|---|---|---|---|---|---|
| | Amount | | | Gas | Gas | Isoamyl | | |
| Experiment | (in | Temperature | Pressure | N₂ | Ar | Alcohol | GHSV | Conversion |
| No. | Volume) | ° C. | [bar] | [%] | [%] | (vol %) | [h⁻¹] | [%] |
| 119 | 1.0 ml | 275 | 1.0039975 | 80 | 10 | 9.9 | 2500 | 4.5255076 |
| 120 | 1.0 ml | 300 | 1.0039975 | 80 | 10 | 9.9 | 2500 | 67.611455 |
| 121 | 1.0 ml | 325 | 1.0039975 | 80 | 10 | 9.9 | 2500 | 0.906006 |
| 122 | 1.0 ml | 350 | 1.0039975 | 80 | 10 | 9.9 | 2500 | 71.652135 |

From Table 19, it is evident that on passing a gas feed of 9.9 vol % isoamyl alcohol at a GHSV of 2500 h⁻¹ over 1.0 ml of (Pd) as catalyst, 0.9% to 71.6% conversion of isoamyl alcohol was achieved at a pressure of 1.0 bar, and at a temperature in the range of 275 to 350° C. The results of Table 19 are presented graphically in FIG. 18. From Experiments 119 to 122, it was observed that the yield of the isobutene was in the range of 1 mol % to 99 mol %.

It is evident from Table 19 and FIG. 18 that (Pd) as catalyst is actively involved in 0.900 to 71% conversion of isoamyl alcohol.

1.-27. (canceled)

28. A method for preparing at least one olefin of formula (I), 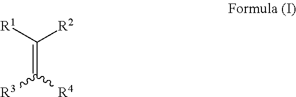
wherein, R¹, R², R³ and R⁴ are independently selected from hydrogen and C₁-C₂₀ alkyl, the method comprising:
a step of contacting at least one alcohol of formula (II), 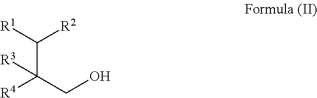
wherein, R¹, R², R³ and R⁴ are as defined above;
with at least one catalyst comprising:
a. at least one catalytically active metal which is Platinum (Pt), wherein the at least one catalyst comprises the at least one catalytically active metal in an amount from 0.005 wt % to 10.0 wt %, with respect to total weight of the catalyst;
b. at least one promoter selected from Tin (Sn), Silver (Ag), Iridium (Ir), Rhenium (Re), or combinations thereof; and
c. at least one carrier on which the catalytically active metal is supported, selected from a non-acidic material, which is zirconia;
at a partial pressure in the range of 0.02 to 4.0 bar, and at a temperature in the range of 200° C. to 450° C.

29. The method according to claim 28, wherein R³ and R⁴ are hydrogen.

30. The method according to claim 28, wherein R¹ and R² are independently selected from hydrogen and C₁-C₂₀ alkyl; and R³ and R⁴ are hydrogen.

31. The method according to claim 28, wherein R¹ is C₁-C₂₀ alkyl, and R², R³ and R⁴ are hydrogen.

32. The method according to claim 28, wherein the at least one alcohol of formula (II) is selected from propanol, butanol, isoamyl alcohol, pentanol, hexanol, heptanol, octanol, nonanol, decanol, or undecanol.

33. The method according to claim 28, wherein the at least one promoter is present in an amount in the range from 0.005 wt % to 5.0 wt %, with respect to total weight of the catalyst.

34. The method according to claim 28, wherein the at least one carrier is present in an amount in the range from 90.0 wt % to 99.995 wt %, with respect to total weight of the catalyst.

35. The method according to claim 28, wherein the at least one catalyst comprises at least one catalytically active metal which is Platinum (Pt), at least one promoter which is Tin (Sn), and at least one carrier which is Zirconia (ZrO₂).

36. The method according to claim 28, wherein the at least one catalyst has a BET surface area in the range from 1 to 500 m²/g, as measured by the Brunauer-Emmett-Teller method.

37. The method according to claim 28, wherein the at least one catalyst has a pore volume in the range from 0.1 to 1.0 ml/g, as determined by Mercury (Hg) Porosimetry.

38. The method according to claim 28, wherein the at least one catalyst has a mean pore diameter, as determined by Mercury (Hg) Porosimetry, in the range from 0.008 to 0.06 μm.

39. The method according to claim 28, wherein the step of contacting is carried out at a temperature in the range from 250° C. to 450° C.

40. The method according to claim 28, wherein the step of contacting involves contacting a feed stream comprising the at least one alcohol of formula (II) in a gas phase.

41. The method according to claim 28, wherein the feed stream is fed at a gas hourly space velocity (GHSV) in the range from 100 h⁻¹ to 15000 h⁻¹.

42. The method according to claim 28, wherein amount of the at least one alcohol of formula (II) in the feed stream is in the range from 2.0 wt % to 100.0 wt % based on the total weight of the feed stream.

43. The method according to claim 28, wherein amount of the at least one alcohol of formula (II) in the feed stream is in the range from 75.0 wt % to 95.0 wt % based on the total weight of the feed stream.

44. The method according to claim 28, wherein the feed stream further comprises at least one inert gas selected from N₂, CO₂, CH₄ or Ar.

45. The method according to claim 28, wherein the at least one olefin of formula (I) is obtained in the form of a product stream.

46. The method according to claim 28, wherein the product stream comprises at least one olefin of formula (I), carbon monoxide and hydrogen.

47. The method according to claim 28, involving a step of separating carbon monoxide and hydrogen from the product stream to obtain the at least one olefin of formula (I).
